(19) United States
(12) Patent Application Publication
(10) Pub. No.: US 2020/0093930 A1
(43) Pub. Date: Mar. 26, 2020

(54) HIGH-THROUGHPUT SYNTHESIS OF BIOMOLECULE-POLYMER CONJUGATES

(71) Applicant: BIOHYBRID SOLUTIONS LLC, Pittsburg, PA (US)

(72) Inventors: Antonina SIMAKOVA, Pittsburgh, PA (US); Alan RUSSELL, Pittsburgh, PA (US); Krzysztof MATYJASZEWSKI, Pittsburgh, PA (US)

(21) Appl. No.: 16/619,633

(22) PCT Filed: Jun. 7, 2018
(86) PCT No.: PCT/US2018/036542

§ 371 Date: Dec. 5, 2019

Related U.S. Application Data
(60) Provisional application No. 62/517,570, filed on Jun. 9, 2017.

(51) Int. Cl. A61K 47/58; C12N 9/96; C08F 220/18; C08F 220/56; C08F 2/38

(52) U.S. Cl. CPC A61K 47/58; C12N 9/96; G01N 21/359; C08F 220/56; C08F 2/38; C08F 220/18

(57) ABSTRACT

Provided herein are methods and systems for concurrently synthesizing and screening a plurality of biomolecule-initiator conjugates and biomolecule-polymer conjugates. Also disclosed are methods of removing oxygen from reaction mixtures and methods of purifying the biomolecule conjugates.

### CROSS-REFERENCE

This application claims the benefit of U.S. Provisional Application No. 62/517,570, filed Jun. 9, 2017, incorporated herein by reference in its entirety.

### BACKGROUND OF THE INVENTION

In recent years, researchers have attempted to take advantage of the unique properties of proteins, in particular biorecognition and structural specificity, for use in new applications. Native proteins may be capable of performing a wide array of functions, yet can be limited in their application due to lack of chemical or thermal stability or decreased activity outside of optimal conditions. Research has focused on methods to generate more stable proteins through the synthesis of protein-polymer conjugates. For example, PEGylated proteins can exhibit improved water solubility relative to the unmodified protein. Modification of proteins may enhance their functionality. Polymer conjugation may open up expanded possibilities for using certain proteins in areas such as industrial catalysis and therapeutics.

### SUMMARY OF THE INVENTION

As such, there is a pressing need to quickly synthesize and screen new biomolecule-polymer conjugates with enhanced properties for use in industrial catalysis and therapeutics. The present disclosure addresses this need in the art through a high-throughput system for the synthesis, screening and optimization of biomolecule-polymer conjugates. This system allows for rapid and automated screening of biomolecule-polymer conjugates.

To date, only low-throughput synthesis and characterization methods have been applied to the preparation of biomolecule-polymer conjugates, limiting development to only few types of polymer modification per protein and relying on stochastic guesswork to select the variants tested. The present disclosure utilizes combinatorial and high-throughput bioconjugation systems that rapidly allow screening of a high number of variants within a week (e.g., approximately 10,000/week). Traditional methods would require several scientists, over the course of a year or more, to create and analyze such a large number of bioconjugates. Thus, one of the major benefits of the methods of the present disclosure is the ability to generate a large library of biomolecule-polymer conjugates with varied polymer coverage density, polymer type, size, composition and architecture, and to match the members of the library to the biomolecule performance in order to identify what kind of polymer modification influences various biomolecule properties. Additionally, application of the subject methods in an iterative manner provides an opportunity to merge discovered properties (e.g., to generate a bioconjugate that is both temperature- and pH-stable) in order to obtain optimal performance for a chosen application. Application of the subject methods allows for development of a more thorough understanding of structure-property relationships between polymers and biomolecules, resulting in development of new and better-performing biomolecule-polymer conjugates.

In certain aspects, the present disclosure provides a method of concurrently synthesizing a plurality of biomolecule-initiator conjugates. The method may comprise (a) providing a biomolecule and a controlled radical polymerization initiator to each reaction chamber in a plurality of reaction chambers, wherein identity of the biomolecule, concentration of the biomolecule, identity of the controlled radical polymerization initiator, and concentration of the controlled radical polymerization initiator are independently selected for each reaction chamber; and (b) maintaining the plurality of reaction chambers under conditions suitable for forming a plurality of biomolecule-initiator conjugates. The method may further comprise simultaneously purifying each biomolecule-initiator conjugate, and optionally evaluating one or more properties of each conjugate either before or after purification, in the plurality of biomolecule-initiator conjugates. Optionally, the method may further comprise mixing the biomolecule and the controlled radical polymerization initiator in at least one of the reaction chambers in the plurality, thereby forming a homogenous mixture. The biomolecule may be a peptide or a protein, such as an enzyme or an antibody. In some embodiments, each reaction chamber contains the same biomolecule.

In practicing any of the subject methods, the controlled radical polymerization initiator may comprise an activated ester, alkyl halide or chain transfer agent. Optionally, the controlled radical polymerization initiator is a compound of Formula (I):

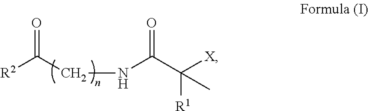

wherein X is a halogen or a chain transfer agent; R¹ is hydrogen or alkyl; R² is an active ester moiety; and n is an integer from 1 to 6. For a compound of Formula (I), X may be Cl, Br or F. Optionally, the controlled radical polymerization initiator is a compound of Formula (II):

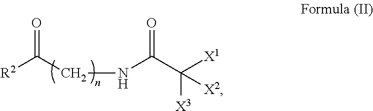

wherein X¹ is halogen or a chain transfer agent; X² is alkyl, aryl, halogen or a chain transfer agent; X³ is hydrogen, halogen or alkyl; R² is an active ester moiety; and n is an integer from 1 to 6. For a compound of Formula (II), X¹ may be Cl, Br or F. In some embodiments, X² is C₁₋₆ alkyl, phenyl, halogen or a chain transfer agent, such as X² is methyl, phenyl, halogen or a chain transfer agent. In some embodiments, X² is Cl, Br or F. In some embodiments, X³ is hydrogen, halogen or C₁₋₆ alkyl.

In practicing any of the subject methods, the concentration of the controlled radical polymerization initiator may be variable across the plurality of reaction chambers. Optionally, the plurality of reaction chambers comprises at least two different radical polymerization initiators. Each reaction chamber may contain the same controlled radical polymerization initiator. Optionally, the biomolecule is immobilized in the reaction chamber.

In certain aspects, the present disclosure provides a method of screening a plurality of biomolecule-polymer conjugates, wherein the method comprises (a) providing a biomolecule-initiator conjugate, a monomer, and a catalyst to each reaction chamber in a plurality of reaction chambers; (b) maintaining the plurality of reaction chambers under controlled radical polymerization conditions suitable for forming a plurality of biomolecule-polymer conjugates; (c) simultaneously purifying each biomolecule-polymer conjugate in the plurality of biomolecule-polymer conjugates; and (d) evaluating one or more properties of the purified biomolecule-polymer conjugates. The controlled radical polymerization conditions may comprise conditions for an atom transfer radical polymerization (ATRP) procedure or a reversible-addition fragmentation chain transfer (RAFT) procedure. Optionally, polymerization is induced by photoirradiation. Each reaction chamber may be irradiated separately, wherein duration and intensity of the photoirradiation is variable across the plurality of reaction chambers. The providing of (a) may further comprise removing oxygen from the plurality of reaction chambers. The providing of (a) may further comprise (i) combining the biomolecule-initiator conjugate and the monomer in a buffer, thereby forming a mixture; (ii) removing oxygen from the mixture; and (iii) generating an active catalyst species in the deoxygenated mixture. In some embodiments, the catalyst maintains catalytic activity in the presence of oxygen. Oxygen may be removed by an enzyme-catalyzed reaction, optionally comprising one or more enzymes selected from glucose oxidase, bilirubin oxidase, catechol dioxygenase, and luciferase.

In some embodiments, a method disclosed herein may further comprise mixing the biomolecule-initiator conjugate, the monomer, and the catalyst in at least one of the reaction chambers in the plurality, thereby forming a homogenous mixture. In some embodiments, a polymer of the biomolecule-polymer conjugate formed by polymerization of the monomer is responsive to stimuli, such as pH, temperature or light. The concentration of monomer, concentration of the catalyst, and/or the identity of the catalyst may be variable across the plurality of reaction chambers. The biomolecule-initiator conjugate may be immobilized in the reaction chamber. The monomer may be selected from a (meth)acrylate and a (meth)acrylamide. In some embodiments, the monomer comprises a mixture of at least two monomers selected from a (meth)acrylate and a (meth)acrylamide. Prior to the purifying of (c), the method may further comprise providing a second monomer to the plurality of biomolecule-polymer conjugates. The second monomer may be selected from a (meth)acrylate and a (meth)acrylamide. A (meth)acrylate and a (meth)acrylamide described herein may comprise one or more of a carboxybetaine, a sulfonate, a quaternary ammonium, a dialkylamino, an amino, a carboxylate, a hydroxyl, a sulfoxy or an oligo(ethylene glycol) moiety. In some embodiments, the monomer comprises a meth(acrylate) or a (meth)acrylamide, wherein the (meth)acrylate or the (meth)acrylamide comprises at least one of a sulfonate anion and an ammonium cation.

Any biomolecule-initiator conjugate described herein may comprise a peptide or a protein. Optionally, the biomolecule-initiator conjugate is a compound of Formula (III):

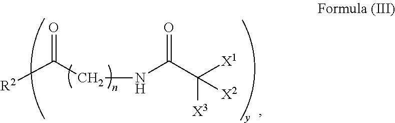

wherein Z is the biomolecule; y is an integer from 1 to 100; X¹ is halogen or a chain transfer agent; X² is methyl, aryl, halogen or a chain transfer agent; X³ is hydrogen, halogen or alkyl; R² is an active ester moiety; and n is an integer from 1 to 6. In some embodiments, X² is methyl, phenyl, halogen or a chain transfer agent.

In practicing any of the subject methods, each reaction chamber in the plurality of reaction chambers may be independently addressable by an automated liquid handling device. Optionally, the plurality of reaction chambers is on a single plate. In some embodiments, the plurality of reaction chambers is on one or more plates. Each reaction chamber on the plate may comprise a membrane at the bottom of the reaction chamber, optionally wherein the membrane is an ultrafiltration membrane. The membrane may be configured to allow continuous fluid delivery through the membrane. In practicing any of the subject methods, the plurality of reaction chambers, optionally a single plate, may comprise at least 24 reaction chambers, such as at least 96 reaction chambers. In practicing any of the subject methods, the purifying may comprise ultrafiltration, optionally wherein the ultrafiltration is vacuum-assisted. In some embodiments, the purifying is accomplished with less than 1 mL of liquid per reaction chamber.

The plurality of reaction chambers may be configured such that absorbance or fluorescence of the purified conjugates can be accurately measured by a spectrophotometer. In certain embodiments, the evaluating comprises ultraviolet-visible spectroscopy, fluorescence spectroscopy or near-infrared spectroscopy. The evaluating may comprise assessing size of the purified conjugates, optionally by one or more of size exclusion chromatography, mass spectrometry and dynamic light scattering. The evaluating may comprise assessing activity, such as enzymatic activity, of the purified conjugates. The enzymatic activity may be assessed under normal working conditions of the biomolecule or under stress conditions. The stress conditions may comprise, relative to the normal working conditions, elevated or reduced temperature, elevated or reduced pH, or an elevated or reduced concentration of water in a buffer solution. A library of biomolecule-initiator conjugates may be prepared according to a method disclosed herein. A library of biomolecule-polymer conjugates may be prepared according to a method disclosed herein. A library of biomolecule-polymer conjugates may be prepared by photoinduced atom transfer radical polymerization. A library of biomolecule-polymer conjugates may be prepared by oxygen-tolerant photoinduced atom transfer radical polymerization.

In certain aspects, the present disclosure provides a method of simultaneously isolating a plurality of bioconjugates from a plurality of reaction mixtures, wherein the method comprises simultaneously passing a plurality of reaction mixtures comprising a plurality of bioconjugates through a plurality of ultrafiltration membranes, wherein the bioconjugates are retained above the membranes, the bioconjugates comprise a biomolecule conjugated to a controlled radical polymerization initiator or a biomolecule conjugated to a synthetic polymer, and wherein each reaction mixture in the plurality is independently purified.

In certain aspects, the present disclosure provides a system for concurrently synthesizing a plurality of biomolecule-polymer conjugates, wherein the system comprises (a) a plurality of reaction chambers configured to hold 1 to 1000 μL of fluid and to allow measurement of absorbance or fluorescence, by a spectrophotometer, of a biomolecule-polymer conjugate contained in each reaction chamber in the plurality; (b) an automated device configured to deliver one or more of a reactant, solvent or catalyst to each reaction chamber in the plurality; (c) optionally, an agitation module configured to mix contents of each reaction chamber in the plurality; (d) a monitoring module configured to monitor progress of a reaction occurring in a reaction chamber in the plurality, wherein the monitoring module is in communication with a spectrophotometer configured to measure at least one of absorbance and fluorescence of the contents of at least one reaction chamber in the plurality; (e) a purification module in fluid communication with the plurality of reaction chambers, wherein the purification module is configured to separate a biomolecule-polymer conjugate from other reaction mixture components, and wherein the other reaction mixture components comprise buffer, monomers and a catalyst; and (f) an evaluation module in visual communication with the plurality of reaction chambers, wherein the evaluation module is configured to assess one or more physical properties of a biomolecule-polymer conjugate contained in each reaction chamber in the plurality. The system may further comprise a photoirradiation module in visual communication with the plurality of reaction chambers, wherein the photoirradiation module is configured to initiate, by photoirradiation, a polymerization reaction in a reaction chamber in the plurality. The photoirradiation module may be configured to separately control the duration of photoirradiation for each of the plurality of reaction chambers. Optionally, the photoirradiation module is configured to separately control the intensity of photoirradiation for each of the plurality of reaction chambers. The system may further comprise a temperature control module configured to maintain the plurality of reaction chambers within a specific temperature range. Optionally, the temperature control module comprises a coolant.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the synthesis of biomolecule-polymer conjugates involving (A) attachment of initiator to biomolecule to form a biomolecule-initiator conjugate and (B) polymerization of monomers to the biomolecule-initiator conjugate to form a biomolecule-polymer conjugate.

FIG. 2A illustrates a combinatorial synthesis of enzyme-initiator conjugates and FIG. 2B illustrates the high-throughput purification and characterization of the conjugates.

FIG. 3A illustrates a combinatorial synthesis, high-throughput purification and characterization of enzyme-polymer conjugates and FIG. 3B illustrates exemplary conjugate libraries.

FIG. 4 illustrates a photomediated ATRP setup, wherein a 96-well plate is placed on top of a light source and optional cooling is provided by fan.

FIG. 5 depicts the reaction progress of polymerization reactions in a 96-well plate photoATRP conversion (top) and the evolution of average molecular weight (closed circles) and polydispersity (open circles) with percent conversion of the same polymerization reactions (bottom).

FIG. 6 provides GPC traces of Reaction A (top) and Reaction C (bottom).

FIG. 7 shows types of chymotrypsin-polymer conjugates prepared by oxygen-tolerant photoATRP.

FIG. 8 describes the formation of NHS-ATRP initiator, its attachment to the surface of an enzyme and subsequent analysis.

FIG. 9 depicts how the number of ATRP initiators attached to an enzyme varies with different reaction conditions (buffer type, pH, ratio of reagents) and influences enzyme activity.

FIG. 10 shows reaction workflow for the modification of lipase with ATRP initiator and an exemplary product analysis assay.

FIG. 11 shows the number of the ATRP initiators attached to lipase depending on the pH of the reaction media and equivalent amounts of NHS-ATRP initiator to the number of available amino groups on the enzyme.

FIG. 12 shows residual enzymatic activity of an enzyme with attached ATRP initiators prepared under varied pH of the reaction media and equivalent amounts of ATRP initiators to the number of available amino groups on the enzyme.

FIG. 13 illustrates the number of attached ATRP initiators on the lipase depending on the concentration of the enzyme and equivalent amounts of NHS-ATRP initiator to number of available amino groups on the enzyme.

FIG. 14 Depicts residual enzymatic activity of the lipase with attached ATRP initiators prepared with different enzyme concentration solutions and equivalent amounts of ATRP initiators to the number of available amino groups on the enzyme.

FIG. 15 shows screening of reaction conditions in a 96-well plate for preparation of the lipase-pNIPAAm conjugate (left) and a final view of the polymerizations in each well (right).

FIG. 16 depicts gel electrophoresis results for lipase-pNIPAAm conjugates prepared under varied reaction conditions.

FIG. 17 illustrates the residual enzymatic activity of bioconjugates of lipase-pNIPAAm prepared under varied reaction conditions.

FIG. 18 depicts structures of different monomers used to prepare lipase-polymer conjugates in high-throughput combinatorial synthesis and screening.

FIG. 19 shows the results of gel electrophoresis of lipase-polymer conjugates in comparison to lipase modified with just ATRP initiating moieties.

FIG. 20 illustrates the activity of lipase and lipase-polymer bioconjugates in catalyzing soybean oil transesterification reactions with methanol (FFA—free fatty acid, FAME—fatty acid methyl ester).

### DETAILED DESCRIPTION OF THE INVENTION

### Definitions

The term “biomolecule” refers to a protein, peptide, enzyme, or antibody. The terms “protein” and “peptide” are used interchangeably herein to refer to a polymer of amino acids.

The term “chain transfer agent” refers to an agent used in polymerization that has the ability to stop the growth of a molecular chain by yielding an atom to the active radical at the end of the growing chain.

The term plurality as used herein may refer to any number greater than 1, such as a number equal to or greater than 2, 6, 12, 24, 48, 96, 192, 384, 768 or 1536.

The term “controlled radical polymerization initiator” refers to a molecule that generates a radical species to begin the synthesis of a polymer chain by successive addition of free-radical building blocks. The terms “controlled radical polymerization initiator” and “initiator” are used interchangeably herein to refer to a molecule that begins a radical polymerization process.

The term “biomolecule-initiator conjugate” refers to a complex that comprises both a biomolecule and one or more controlled radical polymerization initiators, such as five or more, 10 or more, 25 or more, 50 or more, or 100 or more controlled radical polymerization initiators. Preferably, the one or more controlled radical polymerization initiators are covalently attached to the biomolecule.

The term “biomolecule-polymer conjugate” refers to any complex that comprises both a biomolecule and one or more polymer chains, such as five or more, 10 or more, 25 or more, 50 or more, or 100 or more polymer chains. Preferably, the one or more polymer chains are covalently attached to the biomolecule.

The term “atom transfer radical polymerization” (ATRP) refers to a polymerization technique that forms carbon-carbon bonds via a transition metal catalyst. “Reversible-addition fragmentation chain transfer” (RAFT) refers to a polymerization technique that uses a chain transfer agent to give control over the generated polymer weight.

The terms fourier transform infrared, near-infrared, high-performance liquid chromatography, gas chromatography, nuclear magnetic resonance, mass spectroscopy and gel permeation chromatography are referred to as FTIR, NIR, HPLC, GC, NMR, MS, and GPC, respectively. Monomeric 2-(dimethylamino)ethyl methacrylate is referred to as DMAEMA and poly(2-(dimethylamino)ethyl methacrylate) is referred to as PDMAEMA. Low critical solution temperature is referred to as LCST and upper critical solution temperature is referred to as UCST. Chymotrypsin is referred to as CT, poly(quaternary ammonium) is referred to as pQA, and 1,1,4,7,10,10-Hexamethyltriethylenetetramine is referred to as HMTETA. Poly(N-isopropylacrylamide) is referred to as pNIPAm and poly[N,N′-dimethyl (methacryloylethyl) ammonium propane sulfonate] is referred to as pDMAPS. N-hydroxysuccinimide is referred to as NHS and tris-[2-(dimethylamino)ethyl]amine is referred to as Me₆TREN. Oligo(ethylene glycol) monomethyl ether methacrylate is referred to as OEOMA, 2-hydroxyethyl 2-bromoisobutyrate is referred to as HOEBiB, and tris(2-pyridylmethyl)amine is referred to as TPMA. N-[3-(dimethylamino)propyl]acrylamide is referred to as DMAPAAm, and (3-acrylamidopropyl)trimethylammonium chloride) is referred to as qNAAm.

### High-Throughput System

The present disclosure provides a high-throughput system capable of varying reaction conditions, monitoring reaction progress, and evaluating properties of synthesized biomolecule-polymer conjugates. Biomolecule-polymer conjugates are synthesized by conjugating polymers with specific functionality to a biomolecule to form a complex that, ideally, combines the advantages of both the polymer and biomolecule while negating weaknesses of each. The large number of different polymer types available, coupled with a wide range of conjugate functionality, makes it difficult to rapidly synthesize and optimize biomolecule-polymer conjugates for a particular function or application. Additionally, it is possible to graft multiple types of polymers to the same biomolecule, further expanding the number of possible reaction conditions to optimize. These polymers influence the final functionality of the biomolecule-polymer conjugates, including chemical and thermal stability, size, catalytic activity, solubility, and pharmacokinetics.

While modification of biomolecules may be achieved biologically by random mutation, or by cloning/expression, expression systems, biodiversity mining, site directed mutagenesis, or directed evolution, these methods often only yield incremental improvements, are difficult and expensive to scale, require long development times, and often only provide situational solutions. Current synthetic methods involve the slow process of varying and attempting to optimize each reaction parameter in turn. This may include optimization of type and concentration of polymer and biomolecule, reaction time, temperature, and purification steps. This stepwise variation of reaction parameters leads to long periods of synthetic trial and error followed by even more laborious optimization of conditions.

The present disclosure offers a solution by using polymer-based biomolecule engineering and high-throughput synthesis to rapidly screen reaction conditions and assess final biomolecule-polymer conjugate functionality. An automated system of this nature may solve the arduous process of varying numerous parameters and permit exploration of biomolecule-initiator and biomolecule-polymer conjugate synthetic space in parallel, allowing for rapid generation of finished conjugates as well as large amounts of data concerning the effect of reaction conditions on final composition and functionality. Furthermore, a high-throughput system may be programmed with self-learning algorithms to take in data and results from first rounds of synthesis and act as a feedback loop to generate new conditions in an effort to Pareto optimize conjugate synthesis.

A system of this nature for simultaneously synthesizing a plurality of biomolecule-polymer conjugates may comprise: (a) a plurality of reaction chambers configured to hold 1 to 1000 μL of fluid and to allow measurement of absorbance or fluorescence, by a spectrophotometer, of a biomolecule-polymer conjugate contained in each reaction chamber in the plurality; (b) an automated device configured to deliver one or more of a reactant, solvent or catalyst to each reaction chamber in the plurality; (c) optionally, an agitation module configured to mix contents of each reaction chamber in the plurality; (d) a monitoring module configured to monitor progress of a reaction occurring in a reaction chamber in the plurality, wherein the monitoring module is in communication with a spectrophotometer configured to measure at least one of absorbance and fluorescence of the contents of at least one reaction chamber in the plurality; (e) a purification module in fluid communication with the plurality of reaction chambers, wherein the purification module is configured to separate a biomolecule-polymer conjugate from other reaction mixture components, and wherein the other reaction mixture components comprise buffer, monomers and a catalyst; and (f) an evaluation module in visual communication with the plurality of reaction chambers, wherein the evaluation module is configured to assess one or more physical properties of a biomolecule-polymer conjugate contained in each reaction chamber in the plurality.

The system may further comprise a photoirradiation module in visual communication with the plurality of reaction chambers, wherein the photoirradiation module is configured to initiate, by photoirradiation, a polymerization reaction in a reaction chamber in the plurality. The photoirradiation module may be configured to separately control the duration of photoirradiation for each of the plurality of reaction chambers. Optionally, the photoirradiation module is configured to separately control the intensity of photoirradiation for each of the plurality of reaction chambers. A system comprising a photoirradiation module may further comprise a temperature control module, such as a cooling module. The temperature control module may be configured to maintain the plurality of reaction chambers at a specific temperature, or within a specific temperature range. Optionally, the temperature control module comprises a fan. The fan may be placed under the photoirradiation module to provide cooling for the plurality of reaction chambers. If additional cooling is required, the temperature control module may further comprise a coolant. For example, a fan can be placed on top of a coolant to produce a cooler air stream directed toward the plurality of reaction chambers.

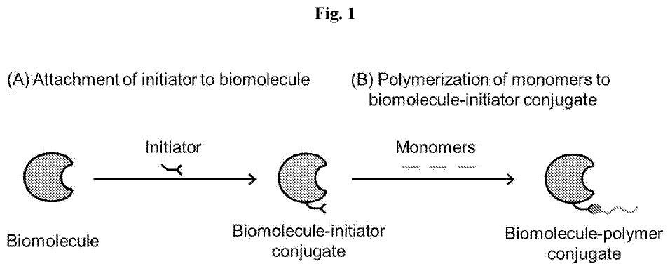

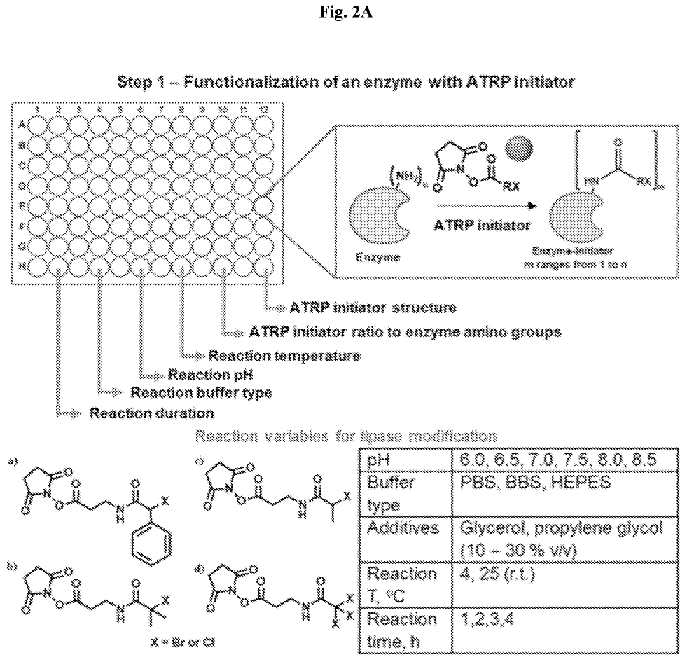

A central part of a high-throughput system of this nature is the ability to explore the synthetic space and variation of both biomolecule-initiator and biomolecule-polymer conjugates (FIG. 1). The synthesis of biomolecule-polymer conjugates involves first attaching initiators to biomolecules of interest to form biomolecule-initiator conjugates. Biomolecule-polymer conjugates may then be formed via polymerization of one or more monomers of interest. Determining and then optimizing advantageous properties of finished biomolecule-polymer conjugates involves varying synthetic conditions for both attaching initiators to biomolecules as well as grafting a polymer onto the biomolecule via a polymerization reaction to form a biomolecule-polymer conjugate. Both synthetic steps may alter the functionality of the resultant biomolecule-polymer conjugates.

### Biomolecules

The first step in the synthesis of a biomolecule-polymer conjugate involves selecting a biomolecule of interest, such as a protein or enzyme. Proteins may be comprised of thousands to fewer than one hundred amino acid residues linked by peptide bonds, linearly and/or branched, and folded in three-dimensional configurations. The configuration of the protein determines function. Exemplary proteins include chymotrypsin, phospholipase A, lipase, nitrilase, acylase, and transaminase. In some embodiments, the biomolecule is an enzyme. Enzymes function as biological catalysts that may increase the rate of a biological reaction, such as by 10⁶ to 10¹⁴ fold. Most enzymes are reactive under mild physiological conditions. The configuration of an enzyme, and therefore, the position of available binding sites, contributes to the specificity and selectivity of the enzyme. Enzymes have an active binding site to receive and bind with a substrate, such as another protein, to form enzyme-substrate complexes. Upon binding, the enzyme catalyzes the relevant reaction to produce the end product of the catalyzed reaction. Enzymes interact with their substrates and targets by removing them from a solvent, binding, reacting and then returning products to solution. Exemplary classes of enzymes include esterases, lipases and proteases. In nature, there are complex interactions that dictate the final protein structure having its specific function. Controlled manipulation of the properties of a biomolecule, and in particular, an enzyme, may expand the scope of applications in which the biomolecule may be used, for example, in therapeutic applications. For example, a biomolecule-polymer conjugate may retain the enzymatic activity of the native biomolecule while having improved stability in a particular solvent, at a given pH, and/or at a specific temperature.

### Polymerization Methods

The overall synthetic approach used to generate biomolecule-polymer conjugates of a particular biomolecule is important in achieving the desired functionality of the conjugate. Polymer conjugation was initially established using a “grafting to” technique, where pre-synthesized, end functionalized polymers are coupled to accessible amino acid side chains or end termini on the protein surface. The grafting site of a functionalized synthetic polymer to a biomolecule surface through a coupling reaction is often a random process in which the density and sites of the grafted polymer cannot be controlled. Once the first polymer chain has “grafted to” the biomolecule surface, steric hindrance will often prohibit further polymer binding to near-by sites on the surface, resulting in a low density of the grafted polymer. Although “grafting to” techniques provide a wide range of polymerization reactions and monomers to select from, a large excess of polymer is often required to overcome steric limitations caused by coupled polymers. Additionally, separation of biomolecule-polymer conjugates from unreacted polymer can prove to be difficult. Thus, an alternative approach was developed for synthesis of biomolecule-polymer conjugates that would allow for higher polymer density and finer site control. This “grafting from” approach starts with modification of a protein with polymer initiator functionalities, which can be further extended by polymerization. This technique leads to high yields of well-defined biomolecule-polymer conjugates and simpler purification, as the conjugate only needs to be separated from small-molecule monomers. Thus, biomolecule-polymer conjugates prepared by the “grafting from” method can be produced with reduced cost and more consistent batch-to-batch composition.

### Biomolecule-Initiator Conjugates

Before grafting any type of polymer to a biomolecule, a biomolecule-initiator conjugate must be synthesized. This entails attaching controlled radical polymerization initiators to the surface of a biomolecule, generally through the use of surface reactive amino acids. These surface reactive amino acid side chains may be covalently coupled to an initiator to synthesize biomolecule-initiator conjugates. One aspect of the present disclosure describes a method of concurrently synthesizing a plurality of biomolecule-initiator conjugates comprising (a) providing a biomolecule and a controlled radical polymerization initiator to each reaction chamber in a plurality of reaction chambers, wherein identity of the biomolecule, concentration of the biomolecule, identity of the controlled radical polymerization initiator, and concentration of the controlled radical polymerization initiator are independently selected for each reaction chamber; and (b) maintaining the plurality of reaction chambers under conditions suitable for forming a plurality of biomolecule-initiator conjugates. These biomolecules and initiators can be mixed within reaction chambers to form a homogenous solution, which may improve product yield or reaction kinetics. This homogenous mixing also ensures that a specific, desired density of initiators may be achieved upon a biomolecule surface. The biomolecules may be solubilized in a buffered solution or reversibly immobilized in the reaction chambers, for example, reversibly immobilized on a bead. The method of concurrently synthesizing a plurality of biomolecule-initiator conjugates may further comprise (c) simultaneously purifying each biomolecule-initiator conjugate in the plurality of biomolecule-initiator conjugates; and optionally (d) evaluating one or more properties of the purified biomolecule-initiator conjugates. The purifying step may be conducted before or after the evaluating one or more properties. In some examples, the biomolecule-initiator conjugates are purified prior to the evaluating. One or more properties of the biomolecule-initiator conjugates may be evaluated without purification.

Different degrees of modification of the biomolecule may be achieved by varying the reaction conditions, including, for example, reaction pH, reaction temperature, buffer type, additives (e.g., glycerol or propylene glycol), reaction time, identity of the biomolecule, concentration of the biomolecule, equivalents of the biomolecule, identity of the controlled radical polymerization initiator, concentration of the controlled radical polymerization initiator, and equivalents of the controlled radical polymerization initiator. These reaction conditions may be independently controlled for each reaction chamber in the plurality of reaction chambers. The degree to which the biomolecule is modified by the initiator at this stage controls the ultimate polymer coverage of the biomolecule surface in the second stage when biomolecule-polymer conjugates are synthesized. The reaction progress can be monitored spectrophotometrically. The efficiency of the biomolecule-initiator conjugate reaction can be assessed using a fluorescamine assay, which allows for the quantification of modified amino groups on the biomolecule. Activity of the biomolecule, such as enzymatic activity, can be assessed on a model reaction utilizing the biomolecule-initiator conjugate. Biomolecule-initiator conjugates that exhibit significantly reduced activity may be discarded prior to grafting a polymer to the conjugates. Optionally, activity of biomolecule-initiator conjugates is assessed, and only biomolecule-initiator conjugates that retain at least 30%, at least 40%, at least 50%, at least 60%, at least 70%, at least 80%, at least 90%, or at least 100% of the original biomolecule activity are reacted with monomers under controlled radical polymerization conditions suitable for forming a plurality of biomolecule-polymer conjugates.

While there are different types of controlled radical polymerization initiators to choose from, the initiator preferably comprises a functional group, such as an active ester, for binding with a surface reactive amino acid on a biomolecule. Exemplary activated esters include an N-hydroxysuccinimide ester, a hydroxybenzotriazole ester, or a 1-hydroxy-7-azabenzotriazole ester. The initiator may be immobilized to one or more amines on the surface of the biomolecule in aqueous solution. These initiators may comprise a functional group that reacts with monomers in a polymerization reaction, such as an alkyl halide or a chain transfer agent. Chain transfer agents can be useful for lowering molecular weights in polymerization reactions. A chain transfer agent may be represented by the general structure:

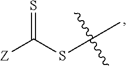

wherein Z is aryl, alkyl, substituted sulfur, substituted oxygen, or substituted nitrogen. Optionally, Z is aryl, heteroaryl, alkyl, substituted sulfur, substituted oxygen, or substituted nitrogen. The chain transfer agent may be any suitable known chain transfer agent used in a RAFT polymerization procedure. Exemplary chain transfer agents include thiocarbonylthiol compounds, such as dithioesters, trithiocarbonates, dithiocarbonates and dithiocarbamates. In one example, the chain transfer agent is cumyl dithiobenzoate.

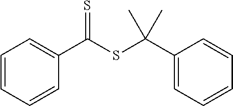 [SMILES: CC(C)(SC(=S)c1ccccc1)c1ccccc1]

In another example, the chain transfer agent is cyanomethyl 1H-pyrrole-1-carbodithioate.

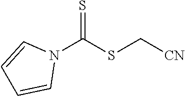 [SMILES: N#CCSC(=S)n1cccc1]

In some examples, the controlled radical polymerization initiator is a compound of Formula (I):

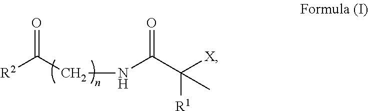

wherein:

X is a halogen or a chain transfer agent;

R¹ is H or alkyl;

R² is an active ester moiety; and

n is an integer from 1 to 6.

Optionally, X is selected from Cl, Br and F. R¹ may be selected from H and C₁₋₆ alkyl, such as methyl, ethyl, propyl and butyl. In some examples, R², together with the carbonyl to which it is attached, forms an active ester moiety selected from an N-hydroxysuccinimide ester, a hydroxybenzotriazole ester, or a 1-hydroxy-7-azabenzotriazole ester.

In some examples, the controlled radical polymerization initiator is a compound of Formula (II):

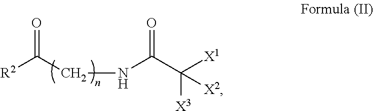

wherein:

X¹ is halogen or a chain transfer agent;

X² is alkyl, aryl, halogen or a chain transfer agent;

X³ is hydrogen, halogen or alkyl;

R² is an active ester moiety; and

n is an integer from 1 to 6.

Optionally, X¹ and X² are independently selected from Cl, Br and F. In some embodiments, X¹ is selected from Cl, Br and F, and X² is selected from C₁₋₆ alkyl, phenyl, halogen and a chain transfer agent. In some embodiments, X² is selected from methyl, phenyl, halogen and a chain transfer agent. X³ may be selected from hydrogen, Cl, Br and F. In some embodiments, X³ is selected from hydrogen, halogen and C₁₋₆ alkyl. In some examples, R², together with the carbonyl to which it is attached, forms an active ester moiety selected from an N-hydroxysuccinimide ester, a hydroxybenzotriazole ester, or a 1-hydroxy-7-azabenzotriazole ester. In some embodiments, X¹ is halogen or a chain transfer agent; X² is methyl, phenyl, halogen or a chain transfer agent; X³ is hydrogen, halogen or C₁₋₆ alkyl; R² is an active ester moiety; and n is an integer from 1 to 6.

In one example, the controlled radical polymerization initiator is an NHS-functionalized amide containing ATRP initiator, such as N-2-bromo-2-methylpropanoyl-β-alanine N′-oxysuccinimide ester:

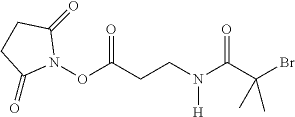 [SMILES: CC(C)(Br)C(=O)NCCC(=O)ON1C(=O)CCC1=O]

In another example, the controlled radical polymerization initiator is N-2-chloro-propanoyl-β-alanine N′-oxysuccinimide ester:

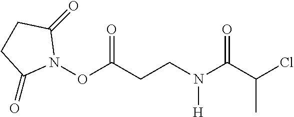 [SMILES: CC(Cl)C(=O)NCCC(=O)ON1C(=O)CCC1=O]

In some examples, R² is selected from:

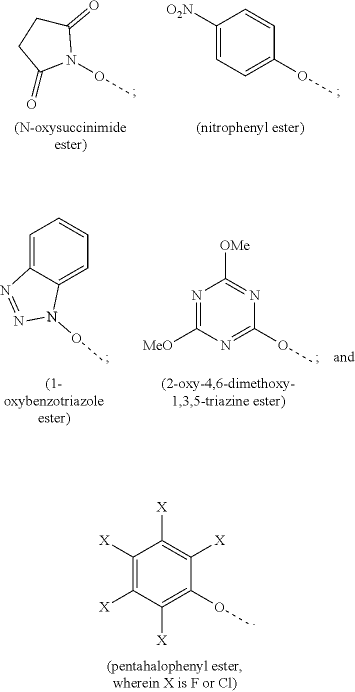

The ratio of amount of initiator to biomolecule during biomolecule-initiator synthesis plays a large role in the properties of biomolecule-polymer conjugates. Using a larger quantity of initiator may lead to higher initiator density on the surface of the biomolecule and better control over the eventual size of the synthesized conjugate while utilizing the “grafting from” conjugation technique.

In one aspect described herein, a biomolecule-initiator conjugate is a compound of Formula (III):

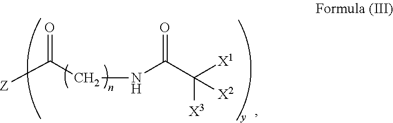

wherein:

Z is a biomolecule;

y is an integer from 1 to 100;

X¹ is halogen or a chain transfer agent;

X² is alkyl, aryl, halogen or a chain transfer agent;

X³ is hydrogen, halogen or alkyl; and

n is an integer from 1 to 6.

Optionally, X¹ is selected from Cl, Br and F. X² and X³ may be selected from hydrogen, methyl, Cl, Br and F. In some embodiments, X¹ is selected from Cl, Br and F and X² and X³ are selected from hydrogen and methyl. In some embodiments, n is an integer from 2 to 3, such as n is 2. In some embodiments, X¹ is selected from Cl and Br, X² and X³ are selected from hydrogen and methyl, and n is 2. In some embodiments, Z is a protein, such as an enzyme. In some embodiments, Z is a biomolecule; y is an integer from 1 to 100; X¹ is halogen or a chain transfer agent; X² is methyl, phenyl, halogen or a chain transfer agent; X³ is hydrogen, halogen or C₁₋₆ alkyl; and n is an integer from 1 to 6.

A high-throughput system may be used to rapidly screen different reaction conditions and initiator to biomolecule ratios to optimize the synthesis of different biomolecule-initiator conjugates. Without the use of an automated system, replicating exact reaction conditions while systematically changing one or more variables is both time consuming and difficult.

### Biomolecule-Polymer Conjugates

The ability of high-throughput systems to efficiently screen synthetic conditions for biomolecule-initiator conjugates also allows for the selection of different monomers during polymerization reactions and “grafting from” the biomolecule-initiator conjugates. The ability to use different monomer types is beneficial in predicting efficacy of polymer conjugation in synthesizing functional biomolecule-polymer conjugates. In one aspect, the present disclosure provides a method of screening a plurality of biomolecule-polymer conjugates comprising (a) providing a biomolecule-initiator conjugate, a monomer, and a catalyst to each reaction chamber in a plurality of reaction chambers; (b) maintaining the plurality of reaction chambers under controlled radical polymerization conditions suitable for forming a plurality of biomolecule-polymer conjugates; (c) simultaneously purifying each biomolecule-polymer conjugate in the plurality of biomolecule-polymer conjugates; and (d) evaluating one or more properties of the purified biomolecule-polymer conjugates. The purifying step may be conducted before or after the evaluating one or more properties. Preferably, the biomolecule-polymer conjugates are purified prior to the evaluating. The polymerization of monomers to the biomolecule-initiator conjugates is an important step in imparting new and unique properties to the final biomolecule-polymer conjugates.

Different degrees of modification of the biomolecule may be achieved by varying the reaction conditions, including, for example, reaction pH, reaction temperature, buffer type, additives (e.g., glycerol or propylene glycol), reaction time, identity of the biomolecule, concentration of the biomolecule, equivalents of the biomolecule, identity of the monomer, concentration of the monomer, equivalents of the monomer, identity of the catalyst, concentration of the catalyst, equivalents of the catalyst, photoirradiation intensity, and photoirradiation duration. These reaction conditions may be independently controlled for each reaction chamber in the plurality of reaction chambers.

As discussed above, utilizing a “grafting from” polymerization technique for polymer attachment to a biomolecule allows for greater control over the properties of the final biomolecule-polymer conjugate. Using this technique, the biomolecule-initiator conjugate is reacted with monomers of choice in a controlled radical polymerization reaction, such as atom transfer radical polymerization (ATRP) or reversible-addition fragmentation chain transfer (RAFT), to grow the polymer chains on each active site where the initiator was immobilized. In practicing any of the methods described herein, the controlled radical polymerization conditions may include ATRP or RAFT polymerization procedures. If ATRP is the polymerization procedure of choice, then the initiator typically comprises a halogen (e.g., one or more of X, X¹, X² and/or X³ in a compound of Formula (I), (II) or (III) is a halogen). If RAFT is the polymerization procedure of choice, then the initiator typically comprises a chain transfer agent (e.g., one or more of X, X¹ and/or X² in a compound of Formula (I), (II) or (III) is a chain transfer agent). The resultant biomolecule-polymer conjugate is preferably a bioactive molecule having desired properties for a typical application. Preferably, the biomolecule-polymer conjugate retains its original function or activity, or a portion thereof, relative to the native biomolecule.

Polymers may be designed to specifically alter the properties of a biomolecule, such as to improve solubility, increase retention in a particular biological environment, alter pH tolerance, or to increase stability of the biomolecule under particular conditions. Many types of polymers may be attached to biomolecules, including water-soluble, zwitterionic, temperature, or pH-responsive polymers. For example, attachment of polyethylene glycol (PEG) polymers to a biomolecule, termed PEGylation, may help hide protein based therapeutics from the immune system by increasing conjugate size to slow elimination from the body. However, little additional specific functionality is added by PEGylation. In contrast, selection of polymers such as polyacrylate can impart new functionality to the resulting conjugate. Acrylate polymers conjugated to proteins not only increase the size of the conjugate but also influence the pH dependence of solubility and activity of the resulting conjugate.

Stimuli responsive monomers, such as (meth)acrylates or (meth)acrylamides, may be used to modify a biomolecule of interest such that the resulting conjugate will exhibit a desired property, and importantly, will function in the manner for which it was designed in a relevant microenvironment. One example of a stimuli responsive monomer is PDMAEMA. PDMAEMA exhibits a phase transfer between super-hydrophilic and hydrophobic characteristics below and above its pKₐ. The chains of PDMAEMA are expanded in aqueous solution when tertiary amine groups of PDMAEMA are protonated and hydrated below the pKₐ. In contrast, the polymer chains are collapsed by deprotonation and dehydration of the amine group above the pKₐ. There are also conformational changes in PDMAEMA below and above its LCST. Selection of monomeric DMAEMA for inclusion in a conjugate system has large influence over the inherent properties of the synthesized final conjugate. Exemplary stimuli responsive monomers include 3-(Dimethylamino)-1-propylamine, N-[3-(Dimethylamino)propyl]acrylamide, and 2-(Dimethylamino)ethyl methacrylate.

While using different polymer types imparts different functionality in the biomolecule-polymer conjugate, these properties stem from the different types of monomers available for polymerization. The four main monomer types used in synthesis of biomolecule-polymer conjugates are uncharged, zwitterionic, cationic, and anionic. Selection of multiple types of monomers in the same synthetic procedure is possible using a high-throughput system and may give rise to unique functionality in the final biomolecule-polymer conjugates. In addition to differing intrinsic monomer functionality, a high-throughput system gives control over varied ratio, concentration, and type of monomers used in conjugate synthesis. For example, one may cycle polymeric growth between two or three different monomers—A, B, and C—in series, giving rise to a single polymer structure that comprises a repeating A-B-C-A-B-C sequence. Alternatively, one can grow three distinct polymers, composed of only A, B and C units, respectively, on the same biomolecule surface to give the conjugate different properties. The use of an automated high-throughput system described herein gives meticulous control over reaction conditions, allowing one to start and/or stop a polymerization reaction with great precision over sequence of monomers used as well as monomeric concentration of each step. This expanded selection and control over reaction parameters allows for the use of copolymers and block copolymers to be grown from the surface of the biomolecule and expands the possible functionalities that can be incorporated into the final conjugate structure.

### Oxygen Removal

High-throughput systems and methods described herein may further comprise removal of oxygen from each reaction chamber in the plurality of reaction chambers during polymerization of monomers to biomolecule-initiator conjugates. Catalytically active species, particularly for polymerization reactions, may become deactivated in the presence of oxygen, thus removal of oxygen from the reaction chambers may allow for greater reaction yield or catalytic efficiency during the polymerization of monomers to biomolecule-polymer conjugates. In one aspect, the present disclosure provides a method of screening a plurality of biomolecule-polymer conjugates, comprising (a)(i) combining a biomolecule-initiator conjugate and a monomer in a buffer in each reaction chamber in a plurality of reaction chambers, thereby forming a plurality of mixtures; (a)(ii) removing oxygen from the plurality of mixtures; and (a)(iii) generating an active catalyst species in the deoxygenated mixtures. In some embodiments, the method further comprises (b) maintaining the plurality of reaction chambers under controlled radical polymerization conditions suitable for forming a plurality of biomolecule-polymer conjugates; (c) evaluating one or more properties of the biomolecule-polymer conjugates; and (d) simultaneously purifying each biomolecule-polymer conjugate in the plurality of biomolecule-polymer conjugates. The plurality of reaction chambers may be maintained in a closed system or a glovebox to reduce oxygen inhibition, or in the presence of oxygen using oxygen tolerant methods (e.g., wherein the polymerization catalyst maintains catalytic activity in the presence of oxygen). The removal of oxygen may be facilitated by an enzyme-catalyzed reaction where said reaction comprises one or more enzymes selected from glucose oxidase, bilirubin oxidase, catechol dioxygenase, and luciferase.

Polymerization reactions described herein may be induced by photoirradiation, optionally in a non-inert environment. The plurality of reaction chambers may be irradiated with UV, violet, or blue light to initiate polymerization. Preferably, the plurality of reaction chambers is irradiated with violet or blue light that is non-destructive to the biomolecules. Photo-induced polymerization further expands the potential monomer and catalyst types available for polymerization and gives access to a wider variety of starting materials for biomolecule-polymer conjugates. Additionally, photo-induced polymerizations may be tolerant to oxygen. In the subject high-throughput systems and methods, parameters such as light intensity and duration may be controlled, optionally separately controlled for each of the plurality of reaction chambers. Photo-induced polymerization reactions can be performed without using a deoxygenating enzyme in a variety of reaction media, including water and buffered solutions (e.g., phosphate buffered saline (PBS), 4-(2-hydroxyethyl)-1-piperazineethanesulfonic acid (HEPES), 2-(N-Morpholino)ethanesulfonic acid hemisodium salt (MES), or phosphate buffer). HEPES buffer contains tertiary amine groups that are beneficial for the reduction of Cu^{II} to Cu^{I} in the presence of light, which may lead to acceleration of photo-induced polymerization reactions.

In one aspect, the present disclosure provides a method of synthesizing a plurality of biomolecule-polymer conjugates, the method comprising (a) providing a biomolecule-initiator conjugate, a monomer, a catalyst, and a buffer to each reaction chamber in a plurality of reaction chambers; (b) inducing controlled radical polymerization by subjecting the plurality of reaction chambers to photoirradiation under an ambient atmosphere comprising oxygen, thereby forming a plurality of biomolecule-polymer conjugates; and (c) simultaneously purifying each biomolecule-polymer conjugate in the plurality of biomolecule-polymer conjugates. The method may further comprise (d) evaluating activity of the purified biomolecule-polymer conjugates relative to the native biomolecule. Preferably, the purified biomolecule-polymer conjugates maintain at least 100% of the activity of the native biomolecule, such as at least 110%, at least 120%, at least 130%, at least 140%, at least 150%, at least 160%, or at least 170%. In some examples, the purified biomolecule-polymer conjugates exhibit one or more properties that increase the activity of the conjugates under stress conditions relative to the native biomolecule. As demonstrated in the Examples, the method of synthesizing a plurality of biomolecule-polymer conjugates is oxygen tolerant and may be performed without deoxygenation, such as without degassing or the addition of a deoxygenating enzyme. Optionally, the providing further comprises addition of trimethylamine (TEA). The initiator may be HOEBiB, iBBr, or Br. In some examples, the buffer is selected from water, PBS and HEPES. Controlled radical polymerization, such as ATRP, is typically induced with violet or blue light, optionally using blue or violet LEDs as the light source, or by addition of reducing agent. Suitable catalysts include copper based catalysts such as CuBr₂, optionally with a ligand such as Me₆TREN or TPMA. In some examples, the monomer is selected from OEOMA, DMAPAAm, DMAEMA, pNIPAAm, QNAAm, TRIS-AAm, DMAAm, AMPSA, AMP, AAm, SBAAm, and HEAAm. The photoirradiation may be completed at any suitable temperature, such as 1-8° C. The method may further comprise (b 1) maintaining the plurality of reaction chambers at a specific temperature range, such as 1-8° C. Preferably, the plurality of reaction chambers is maintained at approximately 4° C.

### Monitoring, Evaluation and Screening

A major advantage in utilizing automated synthetic systems is the integration of in situ reaction monitoring systems to assess reaction progress and/or purity of synthesized materials. A common term for these methods of testing quality as a reaction proceeds is process analytical technology (PAT). Commonly employed in pharmaceutical manufacturing systems, these are automated and integrated technologies, ranging from organic synthesis to spectrometric and chromatographic systems, which are used to assess quality of a final product. PAT methods of the present disclosure typically comprise data analysis, process analytical tools, process monitoring, and continuous feedback. Methods for real-time analysis of various steps may include FTIR spectroscopy for reaction analysis; NIR spectroscopy to measure product uniformity; and HPLC, GC, NMR spectroscopy and MS for reaction analysis and product identity. These techniques may be applied to the synthesis of biomolecule-polymer conjugates, with particular interest being paid to initiator synthesis, attachment of initiators to biomolecules, and tracking of reaction progress, including concentration of monomers during the synthesis of biomolecule-polymer conjugates. For example, proton NMR can be used to conversion, and molecular weight and dispersity can be measured by THF GPC. Gel electrophoresis of biomolecule-polymer conjugates can reveal the amount of unconjugated biomolecule present in a reaction mixture to assess reaction efficiency, and can further be used to assess the size of biomolecule-polymer conjugates.

PAT techniques may also be used in the purification of materials. Purification of conjugates typically comprises increasing the number of biomolecule-initiator or biomolecule-polymer conjugates relative to undesired side products. One aspect of the purification described herein comprises a method of simultaneously isolating a plurality of bioconjugates from a plurality of reaction mixtures. In some embodiments, the method comprising simultaneously passing a plurality of reaction mixtures comprising a plurality of bioconjugates through a plurality of ultrafiltration membranes, wherein the bioconjugates are retained above the membranes, wherein the bioconjugates comprise a biomolecule conjugated to a controlled radical polymerization initiator or a biomolecule conjugated to a synthetic polymer, and wherein each reaction mixture in the plurality is independently purified. The ultrafiltration may be vacuum-assisted. These purification steps and methods may be incorporated into the high-throughput automated system by utilizing the membranes in the plurality of reaction chambers. The membranes may be ultrafiltration membranes that allow small molecules such as water to pass through, but retain larger molecules such as proteins or other biomolecules. This gives the advantage of washing away unattached initiators or monomers from the biomolecule-initiator and biomolecule-polymer conjugates, respectively. An additional advantage of using an automated system is that each reaction chamber may be individually addressable and the ultrafiltration membranes may be configured to allow continuous fluid delivery through the membranes, such as during purification.

Purification of the bioconjugates may be aided by the attachment or immobilization of a biomolecule or a biomolecule-initiator conjugate to a reaction chamber. Flowing fluid through a chamber with an immobilized biomolecule or biomolecule-initiator conjugate may assist in purification, as excess initiators or monomers will be filtered out of the chamber while only the biomolecule-initiator conjugate or biomolecule-polymer conjugate remains. Immobilization methods vary largely with immobilization surface, biomolecule properties, and the desired functionality of the final biomolecule-polymer conjugate. Proteins and other biomolecules may be attached to a surface by one of several different mechanisms. For example, a biomolecule may be attached via passive adsorption, in which the attachment is via hydrophobic interactions or hydrophobic/ionic interactions between the biomolecules and the surface. Covalent immobilization may be used to immobilize a biomolecule to a surface. For example, amine-based covalent linking may be used, utilizing lysine residues on the surface of a biomolecule. Any of these immobilization techniques may be employed during synthesis of biomolecule-polymer conjugates, and it may be found that a specific immobilization technique helps speed up the purification or isolation of biomolecule-initiator and/or biomolecule-polymer conjugates.

Biomolecule-initiator and/or biomolecule-polymer conjugates may be evaluated and/or screened for one or more properties. In some embodiments, the evaluating and/or screening is conducted after purification of the conjugates, though purification may not be required. Utilizing a high-throughput system, reaction chambers can be configured such that absorbance or fluorescence of a reaction mixture or a purified conjugate can be accurately measured by a spectrophotometer. Other evaluation steps may comprise measuring one or more of ultraviolet-visible spectroscopy, fluorescence spectroscopy, near-infrared spectroscopy, and size assessment. Activity, such as enzymatic activity, of biomolecule-initiator and/or biomolecule-polymer conjugates may be assessed under ideal working conditions, then optionally under stress conditions-such as high temperature, extreme pH or various solvent mixtures—to identify conjugates that exhibit improved activity and/or stability relative to the native biomolecule. These varied evaluation techniques help identify and assess desirable properties of conjugates synthesized according to the methods described herein, such as density of initiators on the biomolecule surface, catalytic activity, stability in a particular media and/or condition, as well as degree of biomolecule modification and polymerization. The advantage of using a high-throughput system lies in the fact that conjugates that display one or more properties deemed to be advantageous for a particular application may be easily isolated and similar synthetic space may be explored for biomolecule-initiator and biomolecule-polymer conjugate optimization.

Screening for optimized conditions for polymerization reactions is especially difficult as these reactions are often highly dependent on reaction kinetics. Thus, fast and comparable screening of different polymerization parameters under similar conditions is useful in evaluating many different polymerization conditions. The ability to rapidly screen conditions in parallel allows for direct comparison of different reaction conditions and helps eliminate handling errors, which often affects the results of a kinetic experiment. Automated high-throughput systems allow one to quickly focus on a particularly robust set of reaction conditions and produce biomolecule-initiator or biomolecule-polymer conjugates with a specific function while eliminating reaction conditions that produce conjugates which are unstable or unsuitable for a given application. Small changes in reaction conditions may produce conjugates with vastly different functionality. Thus, parallel screening of reaction conditions is a valuable addition to the synthetic tools available in production of biomolecule-initiator and biomolecule-polymer conjugates.

While parallel screening is an extremely useful tool to explore synthetic conditions and assess function of conjugates, automated feedback loops are useful in generating new synthetic conditions and preparing libraries of biomolecule-initiator and biomolecule-polymer conjugates. These libraries can be narrowly defined for a particular functionality after synthesis of initial conjugates. By screening these initial results and identifying hits for a useful property of interest, a smaller area of synthetic space can be explored and conditions that would not produce usable conjugates can be discarded. This feedback loop is an integral part of rapid parallel screening of conditions that may not initially seem obvious. Additionally, this feedback-loop system, which may comprise a self-learning algorithm, can be repeated for several generations of synthetic optimization for Pareto optimization of biomolecule-polymer conjugates for a given application.

### EXAMPLES

### Example 1

A therapeutic antibody, such as anti-TNF, is modified with a polymer using “grafted from” ATRP. Antibody-polymer conjugates are synthesized by targeted means in an oxygen free environment and are separated from reactants. There currently exists no way that all possible variants of modification density, polymer length and polymer chemistry can be generated in the same reacting system for simultaneous screening of efficacy. In the robotic ATRP high-throughput system, the antibody is simultaneously reacted with hundreds or thousands of variants that systematically probe the synthetic space of modification in a custom designed high-throughput protein polymer synthesis reactor.

The system requires simultaneous separations of reactants from products. Classically, each separation takes liters of dialysis fluids in two long dialysis steps. The custom designed protein-polymer conjugate high-throughput purifier instead uses milliliters of fluid during simultaneous in situ purifications.

Another challenge of miniaturizing and multiplexing the chemistry revolves around the current need to remove all oxygen from the system. High-throughput ATRP synthesis in organic solvents and vacuum systems is typically not compatible with protein ATRP in biologically tolerable conditions. Advanced methods to remove oxygen from the miniature multiplexed reactors are used in this example. In particular, enzymatic oxygen removal chemistries and oxygen tolerant ATRP catalysts are employed. After simultaneously generating 100-10,000 variants of the antibody simultaneously, which vary in the type of initiator, the length and density of polymer and polymer type, high-throughput screening is used to identify the most bioactive variants.

The high-throughput device may be programmed with a self-learning algorithm that would take the first generation of results and select the second generation of optimized conjugates. For instance, the system could find that positively charged polymers are best and simultaneously generate another 1,000-10,000 variants by utilizing the information from the screened experimental data and all previous screenings to deepen and fine tune the set of conjugates generated in the first step. This cycle could continue for multiple generations, with the self-learning algorithm driving refinements at each stage. Finally, after Pareto optimization, a conjugate that cannot be further improved is selected.

### Example 2

In this example, a reversal of chymotrypsin (CT) surface charge using polymer-based protein engineering with pQA, a cationic polymer, is predicted. Other cationic synthetic polymers may be used to both deliver RNA nucleotide based therapies and to enable transport of drugs across the cell membrane. Modification of enzyme surface charge by site directed mutagenesis or synthetic chemistry is shown to cause dramatic effects on protein function. Specifically, modifying protein surface charge is shown to influence the stability and activity profiles of enzymes in non-aqueous solvents, such as ionic liquids, as well as shifting the pH-profile of enzyme activity.

Herein, “grafting from” ATRP to form a high density cationic shell around the chymotrypsin core is predicted. Exogenous chymotrypsin dosing is used to treat pancreatic exocrine deficiency, but low stability due to stomach acid degradation of unmodified chymotrypsin would require higher dosing. The high density cationic pQA shell surrounding chymotrypsin would increase stability, shift the pH profile of chymotrypsin activity, and influence inhibitor binding. Four or more different molecular weight chymotrypsin-pQA conjugates are synthesized to study the effect of polymer-based protein engineering surface charge modification on enzyme kinetics, stability, and inhibitor affinity.

2-(dimethyamino)ethyl methacrylate and bromoethane are added in acetonitrile and stirred overnight. After diethyl ether is added, crystallized QA monomer is filtered off, washed with diethyl ether, and dried. Dynamic light scattering data may be collected to assess particle size before proceeding.

To ensure “grafting from” ATRP of QA monomer from the CT-ATRP initiator conjugate, a solution of QA monomer and CT-initiator conjugate is added to a deoxygenated catalyst solution of HMTETA and copper (I) bromide in de-ionized water, sealed and stirred. CT-pQA conjugates are isolated by dialysis and lyophilized. Cleaved polymer is isolated after acidic hydrolysis and lyophilized. The molecular weight of the cleaved polymer is measured by GPC.

This “grafting from” synthesis may take place in a single or multi plate system with a plurality of reaction chambers and controlled by an automated robotic system. Biomolecules may be immobilized within the reaction chambers and oxygen may be removed from the system. This high-throughput system can simultaneously screen many different synthetic conditions and generate hundreds or thousands of variants of the proposed conjugate. Possible reaction parameters to vary in this example include type of initiator, type of cationic polymer as well as length and density of said polymer. High-throughput screening will help identify conjugates with the most promising functionality, in this case, high stability and rapid enzyme kinetics.

A self-learning algorithm may be programmed into this high-throughput system to calculate optimized synthetic strategies for future generations of synthesized conjugates. The proprietary algorithm analyzes the results from the first generation of synthesized conjugates, selects for a particular feature or functionality—for example, polymers of a specific length that yield higher conjugate stability or a particular polymer density that yields rapid enzyme kinetics—and then uses this information to generate new parameters to introduce into the second generation of synthetic conditions. After multiple iterations of synthetic refinement and Pareto optimization, an optimal biomolecule-polymer conjugate for a particular application is obtained.

### Example 3

Two polymers that show temperature responsiveness are pNIPAm and pDMAPS, though they respond to temperature in sharply distinct ways. pNIPAm exhibits LCST behavior, where above 32° C. the polymer experiences a reversible change in conformation, increasing its hydrophobicity and becoming immiscible in water. The same reversible change is seen for pDMAPS, except that this polymer is immiscible below the UCST. The UCST of pDMAPS exhibits strong dependence on polymer chain length and solution ionic strength while the LCST of pNIPAm is less variable, but is still affected by several factors, such as degree of chain branching and molecular weight.

It is possible to controllably manipulate the kinetics and stability of CT-pDMAPS and CT-pNIPAm bioconjugates using temperature as the trigger for a change in enzyme function. Both pNIPAm and pDMAPS are selected in order to examine changes in relative enzyme activity and stability at stimuli responsive temperatures both above and below room temperature. The contrasting temperature responsive behavior of the UCST and LCST bioconjugates provides an attractive approach to examine how polymer chain collapse at varying temperatures may affect enzyme bioactivity, stability, and substrate affinity.

N-2-bromo-2-methylpropanoyl-β-alanine N′-oxysuccinimide ester is added to CT, dissolved in sodium phosphate buffer and lyophilized to afford a CT-initiator complex.

To synthesize the CT-pDMAPS conjugates, the CT-initiator complex and DMAPS are dissolved in sodium phosphate buffer. In a separate flask, HMTETA and Cu(I)Br are dissolved and added to the DMAPS/CT-initiator complex solution. Lastly, the solution is purified by dialysis and lyophilized.

For CT-pNIPAM synthesis, CT-initiator conjugate and NIPAm are dissolved in deionized water. In a separate flask, Me₆TREN and Cu(I)Br are dissolved and added to CT-pNIPAM solution. Lastly, the solution is purified using dialysis and lyophilized.

Both pDMAPS and pNIPAm are cleaved from the surface of CT using acid hydrolysis. CT-pDMAPS and CT-pNIPAm conjugates are incubated, isolated and lyophilized. Polymer molecular weight is determined using GPC.

CT-pDMAPS and CT-pNIPAm are dissolved in phosphate buffer, CT-pNIPAm samples are heated from 20 to 35° C. and CT-pDMAPS samples are cooled from 30 to 5° C. The absorbance is measured and LCST/UCST temperature calculated from the inflection point on the temperature versus absorbance curves.

This synthesis and temperature responsive testing takes place in a single or multi plate system with a plurality of reaction chambers and controlled by an automated robotic system. Biomolecules may be immobilized within the reaction chambers and oxygen may be removed from the system. This high-throughput system can simultaneously screen many different synthetic conditions and generate hundreds or thousands of variants on the proposed conjugates. Possible reaction parameters to vary include type of initiator, type of polymer as well as length and density of selected polymer. High-throughput screening helps identify conjugates with the most promising functionality, for example response at a specific temperature or high enzymatic activity.

A self-learning algorithm may be programmed into the high-throughput system to calculate optimized synthetic strategies for future generations of synthesized conjugates. The algorithm may analyze the results from the first generation of synthesized conjugates, select for a particular feature or functionality—for example, polymers that are responsive at a specific temperature—and use this information to generate new parameters to introduce into the second generation of synthetic conditions. After multiple iterations of synthetic refinement and Pareto optimization, an optimal biomolecule-polymer conjugate for this particular application should be obtained.

### Example 4

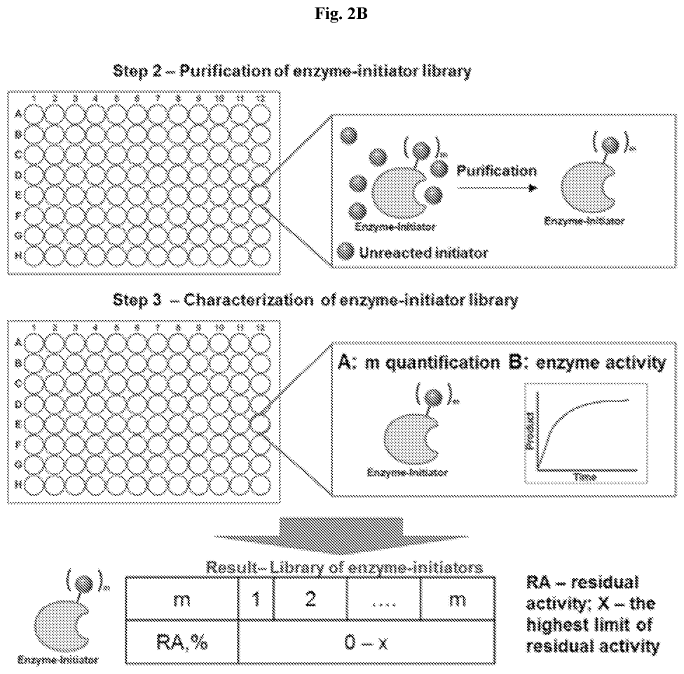

High-throughput combinatorial biomolecule-polymer conjugate synthesis and characterization is achieved by subjecting biomolecules to step-wise functionalization, purification and characterization cycles (FIGS. 2A and 2B). In the first stage, various polymerization initiating moieties are conjugated to a biomolecule and the effect of conjugation on biomolecule performance is tested. The biomolecule is either solubilized in a buffered solution or reversibly immobilized on a bead. Different degrees of modification are achieved by varying reaction conditions resulting in control over final polymer coverage of the biomolecule surface. Upon completion, the synthesized biomolecule-initiator conjugates are purified by high-throughput vacuum assisted (ultra)filtration or other protein purification methods. Enzymatic activity of ATRP initiator-modified enzymes is analyzed on a model reaction, where reaction progress can be detected spectrophotometrically.

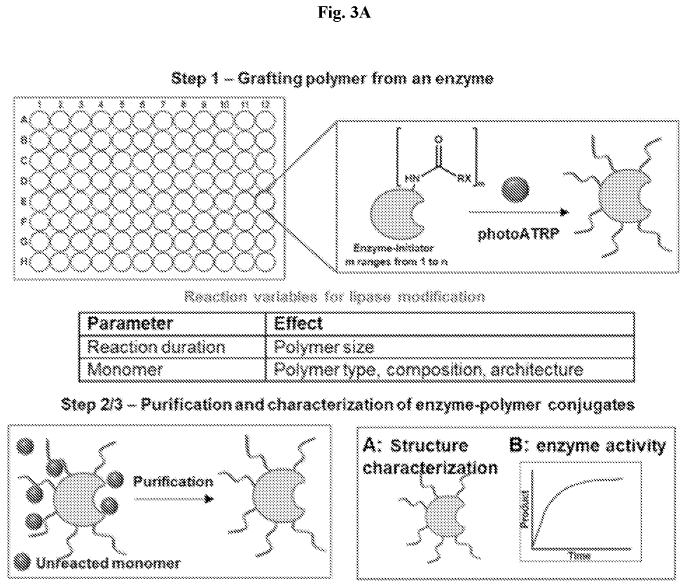

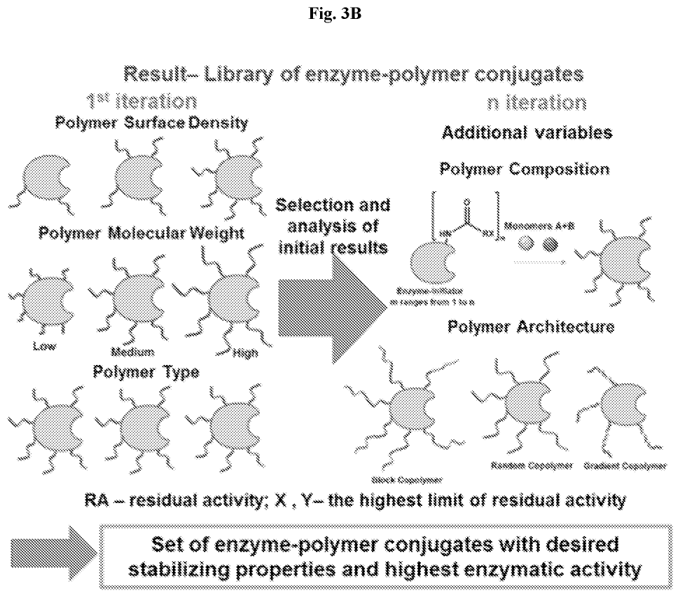

A library of chemically modified biomolecule-initiator conjugates is moved to the second stage, where selected polymers are grafted directly from the biomolecules (FIGS. 3A and 3B). High-throughput polymerization can be performed in closed vials and/or a glovebox to reduce oxygen inhibition (for radical polymerizations), or using oxygen tolerant methods. Photoirradiation can induce polymerization (photoATRP) and can be successfully performed in a 96-well plate under a non-inert environment. PhotoATRP is induced by violet or blue light nondestructive to the biomolecules in 96-well plates (FIGS. 3A and 3B). This method is oxygen tolerant and can be efficiently applied to polymerization in 96-well plates. Upon completion of polymerization, biomolecule-polymer conjugates are subjected to purification from the polymerization catalyst and other reagents. Enzymatic activity of the purified samples is assessed under ideal enzyme working conditions, then under stress conditions (such as incubation under high temperature or low pH) to identify biomolecule-polymer conjugates that exhibit improved properties. This combinatorial photo-ATRP system allows for rapid screening of nearly 10,000 variants with just 100 plates.

### Example 5

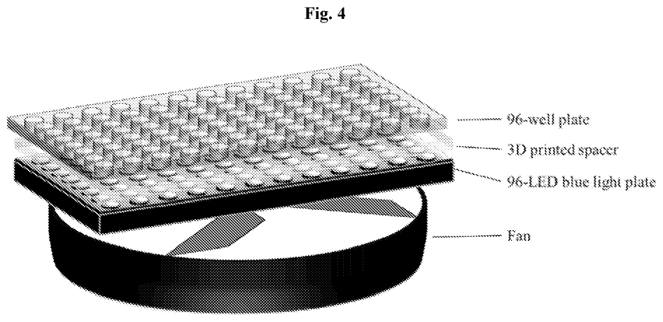

ATRP is induced by addition of reducing agents, electrical current or photoirradiation. Each of these methods generates active catalytic species in situ to initiate polymerization. The setup illustrated in FIG. 4 was used to conduct a photomediated ATRP (photoATRP) process, where a 96-well plate with polymerization solution was placed on top of the light source. If placed directly on the light source, the polymerization solutions may be heated, which may negatively influence stability of biomolecules in the solutions. Thus, optional cooling can be provided by a fan placed underneath the light source. If additional cooling is required, the fan can be placed on top of a coolant in order to blow a cooler air stream, resulting in more efficient cooling. A set of experiments demonstrated that deoxygenation of a polymerization mixture was not necessary in order to prepare polymers by photoATRP (Table 1). A model polymerization reaction in water was utilized to investigate the effect of oxygen on photoATRP:

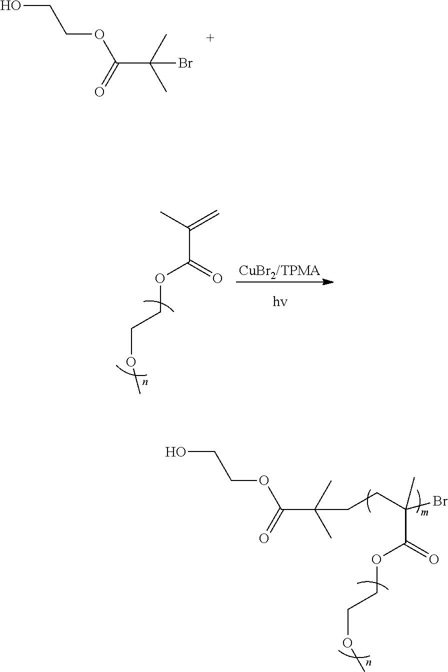

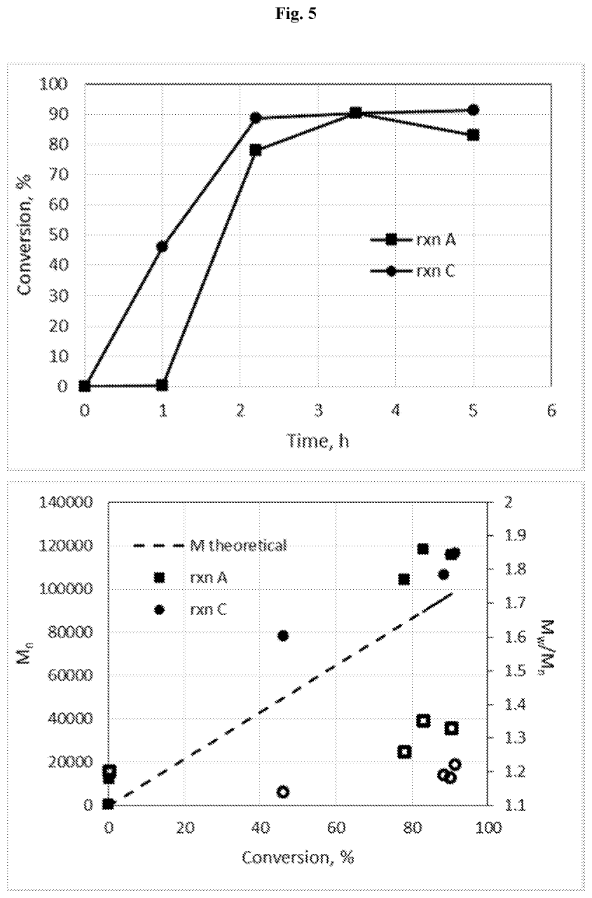

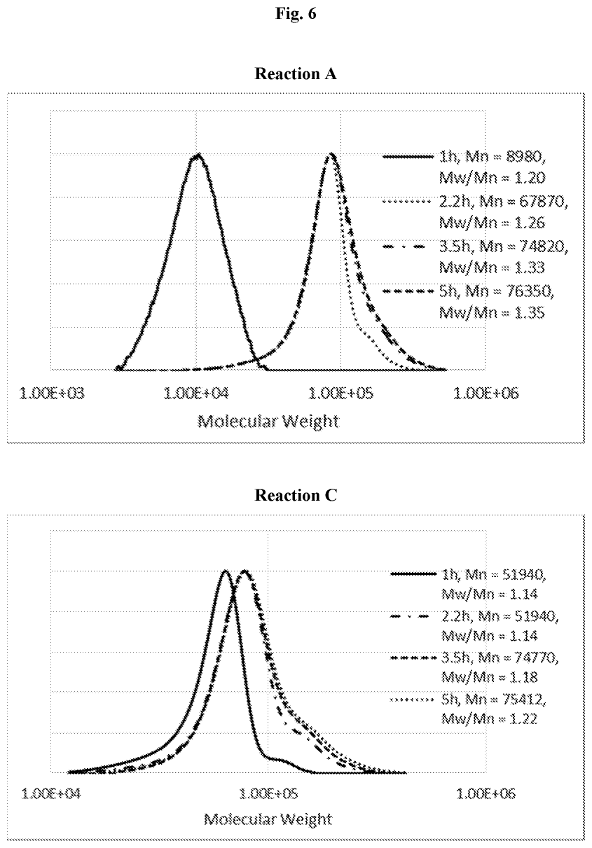

Polymerization was conducted in solutions deoxygenated by argon purging and non-deoxygenated solutions. Polymerization in the presence of oxygen (Reaction A, Table 1) experienced an induction period, but final conversion was almost as high as polymerization in a deoxygenated solution (Reaction C, Table 1). FIG. 5 illustrates that conversion increased with time for both reactions. A linear increase of molecular weight (FIG. 5) and narrow molecular weight distributions (<1.4, FIG. 6) indicated a high level of control over polymerization.

TABLE 1
96-well plate photoATRP induced by violet light:
conditions and results

| | M/I/ | | Time, | Conv., | | | M_{w}/ |
|---|---|---|---|---|---|---|---|
| # | CuX₂/L | Degassing | h | % | Mₜₕ | Mₙ | Mₙ |
| A | 216:1:3:12 | No | 5 | 83 | 89,660 | 118050 | 1.35 |
| C | 216:1:3:12 | Yes | 5 | 91 | 98,770 | 116500 | 1.22 |

M-monomer OEOMA, I-initiator HOEBiB, [HOEBiB] = 2 mM, [OEOMA₅₀₀] = 432 mM (20 vol. %), water, X—Br, L—Me₆TREN.
Conversion was measured by proton NMR.
Molecular weight and dispersity was measured by THF GPC with universal calibration.

TABLE 2
PhotoATRP on 96-well plate irradiated with blue light

| # | M | Degassing | Solvent | Time, h | Conv., % | Mₙ | M_{w}/Mₙ |
|---|---|---|---|---|---|---|---|
| 1 | OEOMA | No | H₂O | 2 | 88 | 119970 | 1.45 |
| 2 | OEOMA | No | PBS | 2 | 95 | 113330 | 1.17 |

Conditions: [OEOMA₅₀₀]:[HOEBiB]:CuBr₂]:[TPMA] = 216:1:3:12, [HOEBiB] = 2 mM, [OEOMA₅₀₀] = 432 mM (20 vol. %).
Conversion was measured by proton NMR.
Molecular weight and dispersity was measured by THF GPC with universal calibration.

Polymerization was conducted by irradiation of different light wavelengths including violet (Table 1) and blue (Table 2). Light was delivered to each of the reaction chambers, while parameters such as light intensity and duration can be controlled for individual chambers.

This method of oxygen-tolerant photoATRP (Table 3, Entries 1-4) was compared to photoATRP performed in the presence of deoxygenating enzyme Glucose Oxidase (GOX) (Table 3, Entries 5-7). Glucose Oxidase consumes oxygen in the presence of glucose, acting as an efficient oxygen removal method. PhotoATRP was successfully performed without using a deoxygenating enzyme in a variety of reaction media, including water and buffered solutions like phosphate buffered saline (PBS) (Table 3, Entries 1-3). Reaction conversion after 2 hours reached ˜40-100% depending on reaction media. Reaction acceleration was detected in the presence of 300 mM HEPES buffer, which contains tertiary amino groups beneficial for the reduction of Cu^{II} to Cu^{I} in the presence of light, according to the following reaction scheme:

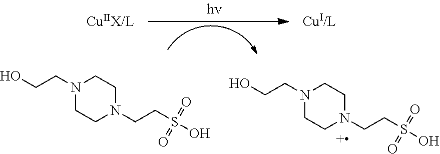 [SMILES: O=S(=O)(O)CCN1CCN(CCO)CC1 ; O=S(=O)(O)CCN1CCN(CCO)CC1]

PhotoATRP in the presence of GOX was only faster in PBS, but similar conversions were reached in water and HEPES (Table 3, Entries 5-7). This set of reactions indicated that photoATRP is an oxygen-tolerant method that typically does not require additional deoxygenation methods.

TABLE 3
Comparison of photoATRP performed in the
presence or absence of degassing enzyme

| | | | | Time, | Conv., |
|---|---|---|---|---|---|
| # | M/I/CuBr₂/L/TEA | Degassing | Solvent | h | % |
| 1 | 432:1:3:12:0 | No | H₂O | 2 | 70 |
| 2 | 432:1:3:12:0 | No | PBS | 2 | 39 |
| 3 | 432:1:3:3:60 | No | H₂O | 2 | 63 |
| 4 | 432:1:3:3:60 | No | 300 mM HEPES | 2 | 99 |
| 5 | 432:1:3:12:0 | GOX | H₂O | 2 | 73 |
| 6 | 432:1:3:12:0 | GOX | PBS | 2 | 81 |
| 7 | 432:1:3:12:0 | GOX | 300 mM HEPES | 2 | 100 |

[HOEBiB] = 1 mM, [OEOMA₅₀₀] = 432 mM (20 vol. %), TEA - triethylamine, L - TPMA; light source - blue LED; conversion was measured by proton NMR.

Different types of monomers, such as (meth)acrylates and (meth)acrylamides, have been successfully polymerized using this method. For example, N-[3-(Dimethylamino)propyl]acrylamide can be photopolymerized according to the reaction scheme:

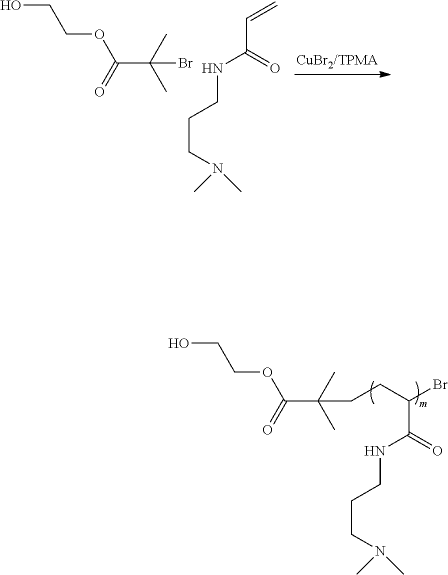

Addition of GOX into the polymerization solution resulted in faster polymerization in water or PBS as the reaction media (Table 4, Entries 1-2 vs. Entries 4-5), but addition of HEPES resulted in >40% conversion within 2 hours, even without addition of GOX.

TABLE 4
PhotoATRP of acrylamide.

| | | | | Time, | Conv., |
|---|---|---|---|---|---|
| # | M/I/CuBr₂/L/X | Degassing | Solvent | h | % |
| 1 | 1217:1:3:3:0 | No | H₂O | 2 | 0 |
| 2 | 1217:1:6:24:0 | No | PBS | 2 | 0 |
| 3 | 1217:1:3:3:0 | No | 300 mM HEPES | 2 | 44 |
| 4 | 1217:1:3:3:0 | GOX | H₂O | 2 | 36 |
| 5 | 1217:1:3:3:0 | GOX | PBS | 2 | 48 |
| 6 | 1217:1:3:3:0 | GOX | 300 mM HEPES | 2 | 74 |

[HOEBiB] = 1 mM; [DMAPAAm] = 1217 mM (20 vol. %), X - TEA, L - TPMA; light source - blue LED; conversion was measured by proton NMR.

### Example 6

The high-throughput photoATRP method described in Example 5 was further applied to the synthesis of biomolecule-polymer conjugates where an initiator was attached to a biomolecule. The biomolecule-initiator conjugate was added to a solution containing a monomer and exposed to light to induce photoATRP. Different reaction chambers were exposed to different wavelengths of light for different lengths of time, and the resulting biomolecule-polymer conjugates were assessed for properties such as chemical and thermal stability, as well as catalytic activity. A high-throughput system was used to iterate through this synthetic cycle multiple times using different monomers as a way of optimizing the properties of the final biomolecule-polymer conjugates. Different types of monomers were polymerized to create a diverse range of biomolecule-polymer conjugates.

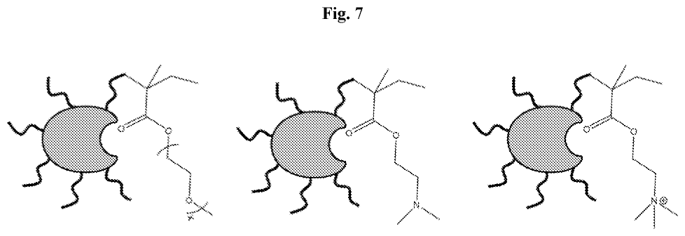

Table 5 shows how several polymer conjugates of chymostrypsin (CT) were prepared simultaneously by photoATRP (FIG. 7). Monomer type and reaction time were varied to prepare a set of different chymotrypsin-polymer conjugates. Measurement of residual activity showed that cationic polymer types were more beneficial for CT activity, increasing its residual activity up to 170% in comparison to native CT (100% residual activity).

TABLE 5
Chymotrypsin-polymer conjugates prepared
by oxygen-tolerant photoATRP

| | | | | Anticipated | |
|---|---|---|---|---|---|
| | Conjugate | Reaction | Conv., | MW of a single | Residual |
| # | Type | time, h | % | polymer chain | activity, % |
| 1 | CT-OEOMA | 2.7 | 20 | 43,200 | 104.7 |
| 2 | CT-OEOMA | 4 | 83 | 224,100 | 24.7 |
| 2 | CT-DMAEMA | 2.7 | 72 | 60,000 | 144.8 |
| 3 | CT-qDMAEMA | 2.7 | 13 | 14,600 | 125.0 |
| 4 | CT-qDMAEMA | 4 | 51 | 57,300 | 168.8 |

[CT-(iBBr)_{6.2±0.2}] = 8.7 mg/ml, [M] = 432 mM; blue LED photoATRP was used to prepare conjugates; conversion was measured by proton NMR.

### Example 7

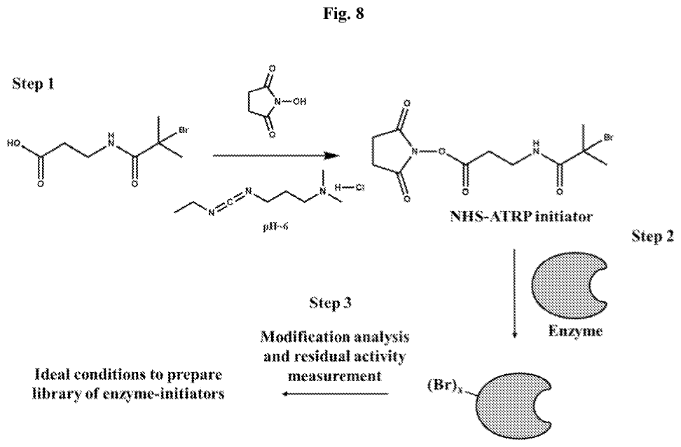

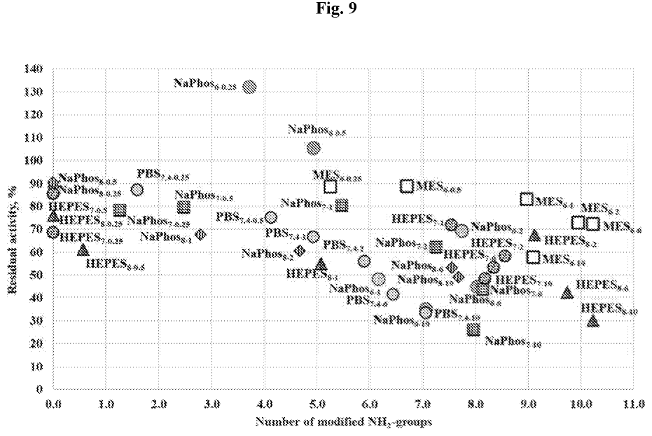

This example describes high-throughput modification of an enzyme with ATRP initiators. Table 6 shows what parameters were varied to identify an ideal set of conditions to prepare diverse structures of an enzyme with attached polymerization moiety (CT-Br in this example). FIG. 8 illustrates that an NHS-ATRP initiator can be prepared beforehand or generated in situ and added to the enzyme under varied conditions. FIG. 9 shows the number of modified amino groups in the enzyme vs. residual activity of the prepared enzyme-initiator (CT-Br). The ideal set of modification conditions where a broad range of modifications can be achieved with maximum retained residual enzymatic activity was selected by evaluating all reaction conditions. For example, Table 7 summarizes the selection of NHS-ATRP initiator attachment conditions where the number of modified enzyme amino-groups ranges from 1.6 to 10.2, and enzymatic residual activity does not drop below 70%. These modified enzymatic samples can be further utilized to prepare enzyme-polymer conjugates as in Example 6.

TABLE 6
Variable parameters in preparation of chymotrypsin-Br (CT-Br)

| | NHS-ATRP | | | |
|---|---|---|---|---|
| | initiator | | | |
| | ratio to | | | |
| # | amino-groups | pH | Buffer | T, ° C. |
| 1 | 0.25 | 6 | Phosphate (6, 6.5, | 4 |
| | | | 7, 7.5, 8) | |
| 2 | 0.5 | 7 | PBS | |
| 3 | 1 | 8 | MES (6) | |
| 4 | 2 | | HEPES (7, 8) | |
| 5 | 6 | | | |
| 6 | 10 | | | |

TABLE 7
Selection of the ATRP initiator immobilization conditions

| | Number of | | |
|---|---|---|---|
| | attached | Residual | |
| # | Br-moieties | activity, % | Conditions |
| 1 | 1.6 ± 0.3 | 87 ± 7 | PBS, pH ~7.3, R0.25 |
| 2 | 3.7 ± 0.3 | 132 ± 6 | Na Phos, pH ~6, R0.25 |
| 3 | 4.9 ± 0.3 | 105 ± 8 | Na Phos, pH ~6, R0.5 |
| 4 | 6.7 ± 1.0 | 89 ± 5 | MES, pH ~6, R0.5 |
| 5 | 9.0 ± 0.3 | 83 ± 3 | MES, pH ~6, R1 |
| 6 | 10.2 ± 0.1 | 72 ± 7 | MES, pH ~6, R6 |

### Example 8

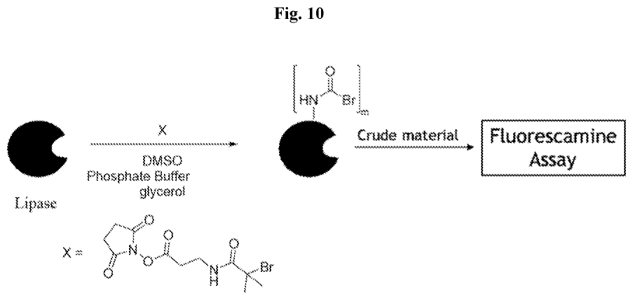

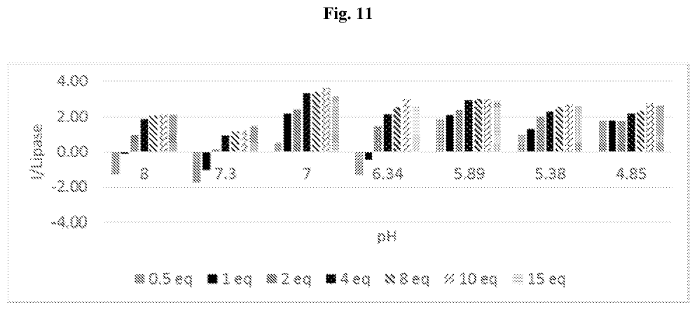

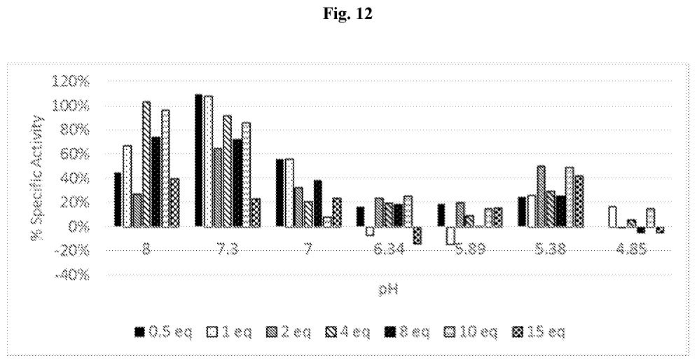

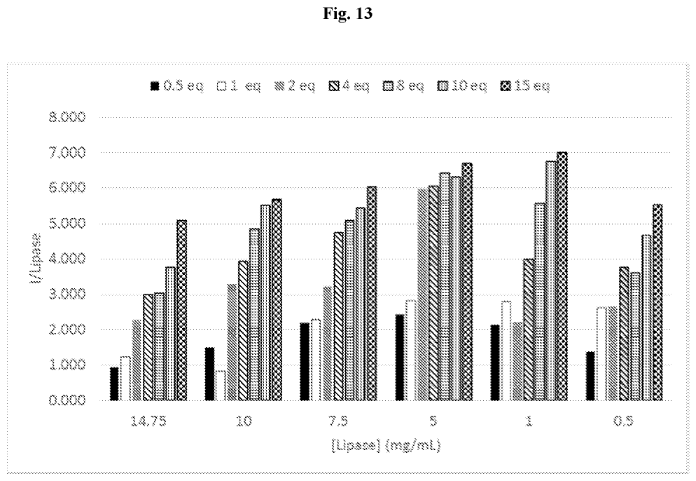

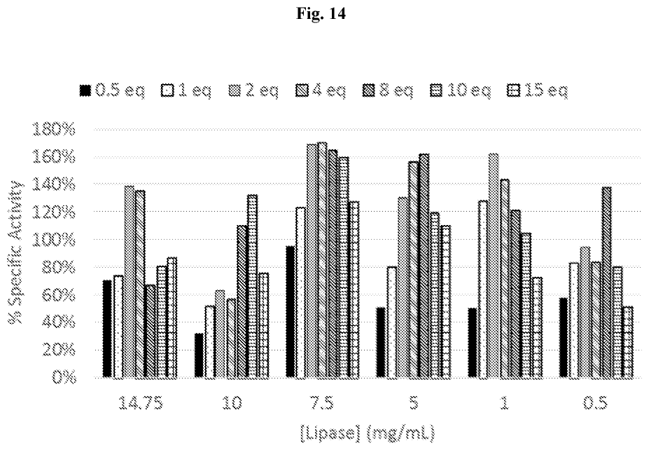

This example illustrates that ATRP initiator attachment screening is an important step to identify the most favorable conditions for making a biomolecule-initiator conjugate having varied amounts of ATRP initiators with preserved activity, such as enzymatic activity. This set of experiments was performed with another enzyme (lipase instead of chymotrypsin) to show the broad applicability of this method. FIG. 10 depicts a reaction workflow, where the ATRP initiator attachment reaction was followed by analysis of the efficiency of the performed reaction by a fluorescamine assay. This assay allows for the quantification of modified amino groups in the enzyme. FIG. 11 shows that both pH of the reaction and number of equivalents of ATRP initiator activated ester influenced the number of modified amino groups. In this example, low equivalents of the ATRP initiator activated ester (below 1) did not result in an efficient reaction, and higher equivalent amounts were required to achieve modification of amino groups in this lipase. Highest efficiency of the modification was achieved at the pH-7 (FIG. 11), and highest preserved activity of the modified lipase was achieved at pH-8 (FIG. 12). In the next set of conditions, the concentration of the enzyme was varied together with the number of equivalents of ATRP initiator activated ester (FIG. 13). FIGS. 13 and 14 demonstrate that the highest level of control of initiator attachment and highest preserved activity of the enzyme was achieved at the concentration of 7.5 mg/ml. Thus, it is evident that screening of the reaction conditions for stage 1 (FIGS. 2A and 2B) is important to identify how number and conditions of the attachment of ATRP initiators to an enzyme influences residual activity of the enzyme.

### Example 9

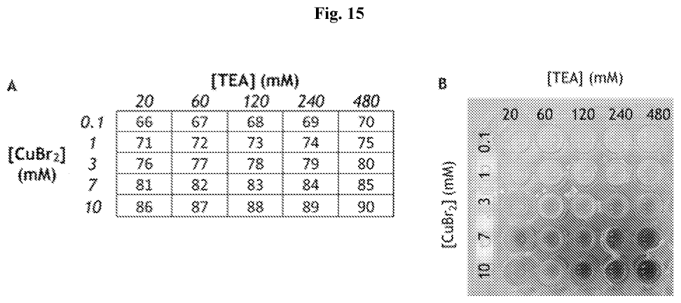

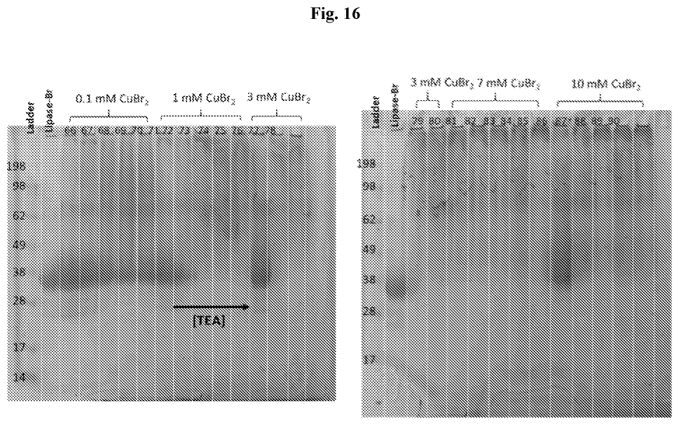

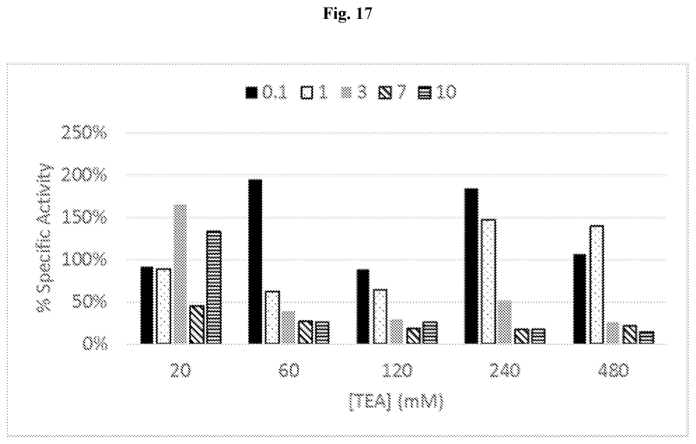

The selected 96-well plate format was shown to be very suitable for conducting polymerization reactions resulting in formation of biomolecule-polymer conjugates. FIG. 15 illustrates that high-throughput bioconjugation can be used to screen polymerization conditions for the desired biomolecule-polymer conjugate. In this example, poly(N-isopropylacrylamide) (pNIPAAm) was grafted from lipase under 25 varied combinations of the polymerization catalytic system (copper bromide and triethylamine (TEA)). As shown in FIG. 15B, higher amounts of copper (I) bromide were produced at the higher copper (II) bromide (7-10 mM) and TEA concentrations (120-240 mM), indicated by the darker color of the wells. Gel electrophoresis analysis of the synthesized biomolecule-polymer conjugates (FIG. 16) also supported that reactions with higher amounts of copper (I) bromide resulted in formation of larger biomolecule-polymer conjugates, as evidenced by a lack of initial material (enzyme). FIG. 17 depicts residual activity of the synthesized biomolecule-polymer conjugates.

### Example 10

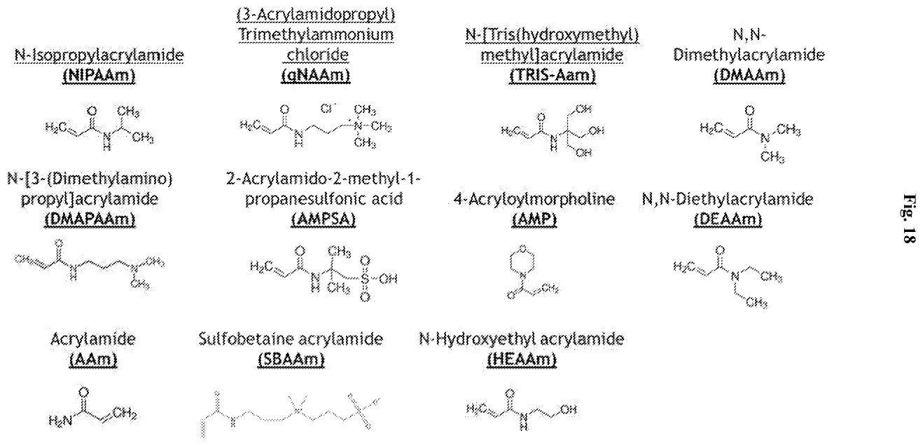

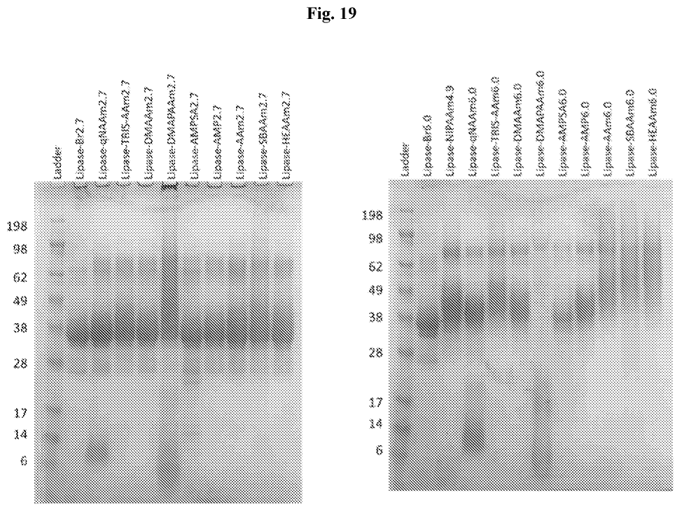

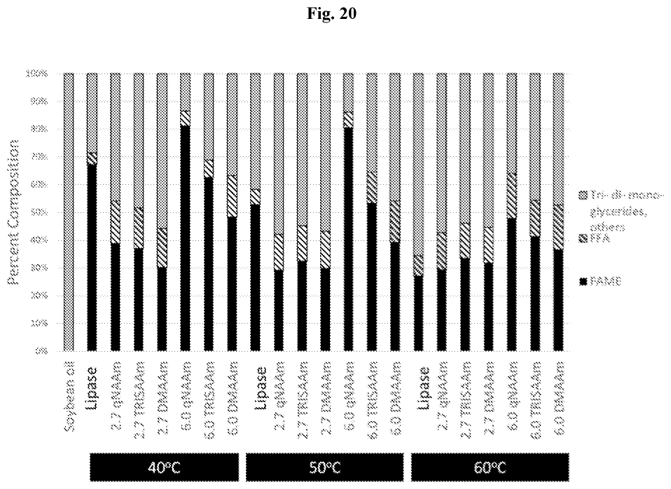

In this example, a diverse set of polymers (FIG. 18) was simultaneously grafted from lipase with varied amounts of attached ATRP initiators using photoATRP procedures in 96-well plates. Tables 8 and 9 describe the type of lipase-initiator, polymer type, polymerization conditions, and size and activity of the biomolecule-polymer conjugates. FIG. 19 shows a shift in a gel electrophoresis assay, indicating successful polymerization. Synthesized biomolecule-polymer conjugates were further assayed for their residual activity (Tables 8 and 9), demonstrating that a majority of the samples retained enzymatic activity. Several samples were further tested for their performance in soybean oil transesterification reactions with methanol. This type of reaction imitates more harsh industrial conditions with higher temperatures and in the presence of organic solvent, which is useful for further selection (FIG. 20). This test revealed that lipase-(qNAAm)₆ (qNAAm is (3-acrylamidopropyl)trimethylammonium chloride) outperformed native lipase and other biomolecule-polymer conjugates, showing improved stabilization under the reaction conditions (temperature, organic solvent).

TABLE 8
Lipase-polymer bioconjugates with 2.7 attached ATRP
initiators. Reaction conditions: 1 hr, blue LED,
4° C., HEPES; M/I/CuBr2/L/TEA = 1333/1/10/10/200

| | Monomer | Radius | Activity |
|---|---|---|---|
| # | Type | (nm) | (umoles/min/mg) |
| 1 | none | 5.1 | 21.2 |
| 2 | qNAAm | 6.9 | 13.5 |
| 3 | TRIS-AAm | 6.1 | 14.1 |
| 4 | DMAAm | 3.5 | 17 |
| 5 | DMAPAAm | 7.5 | 15.8 |
| 6 | AMPSA | 42.1 | 2.2 |
| 7 | AMP | 5.1 | 30.3 |
| 8 | AAm | 3.8 | 29.4 |
| 9 | SBAAm | 3.1 | 27.7 |
| 10 | HEAAm | 3.7 | 31.1 |

TABLE 9
Lipase-polymer bioconjugates with 6 attached ATRP
initiators. Reaction conditions: 1 hr, blue LED,
4° C., HEPES; M/I/CuBr2/L/TEA = 1333/1/10/10/200

| | Monomer | Radius | Activity |
|---|---|---|---|
| # | Type | (nm) | (umoles/min/mg) |
| 1 | none | 5.2 | 26.5 |
| 2 | qNAAm | 52.3 | 6.8 |
| 3 | TRIS-AAm | 6.5 | 6.4 |
| 4 | DMAAm | 5.8 | 8.3 |
| 5 | DMAPAAm | 13.9 | 0.3 |
| 6 | AMPSA | 8.3 | 1.4 |
| 7 | AMP | 5.2 | 10.6 |
| 8 | AAm | 7.3 | 5.5 |
| 9 | SBAAm | 4.9 | 2.7 |
| 10 | HEAAm | 5.1 | 9.1 |

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (i.e., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The articles “a” and “an” are used herein to refer to one or to more than one (i.e., to at least one) of the grammatical object of the article. By way of example, “an element” means one element or more than one element.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of concurrently synthesizing a plurality of biomolecule-initiator conjugates, the method comprising:
(a) providing a biomolecule and a controlled radical polymerization initiator to each reaction chamber in a plurality of reaction chambers, wherein identity of the biomolecule, concentration of the biomolecule, identity of the controlled radical polymerization initiator, and concentration of the controlled radical polymerization initiator are independently selected for each reaction chamber; and
(b) maintaining the plurality of reaction chambers under conditions suitable for forming a plurality of biomolecule-initiator conjugates.

2. The method of claim 1, further comprising simultaneously purifying each biomolecule-initiator conjugate in the plurality of biomolecule-initiator conjugates.

3. The method of claim 2, further comprising evaluating one or more properties of the purified biomolecule-initiator conjugates.

4. The method of claim 1, further comprising evaluating one or more properties of each biomolecule-initiator conjugate in the plurality of biomolecule-initiator conjugates.

5. The method of any one of the preceding claims, further comprising mixing the biomolecule and the controlled radical polymerization initiator in at least one of the reaction chambers in the plurality, thereby forming a homogenous mixture.

6. The method of any one of the preceding claims, wherein the biomolecule is a peptide or a protein.

7. The method of claim 6, wherein the biomolecule is an enzyme or an antibody.

8. The method of any one of the preceding claims, wherein the controlled radical polymerization initiator comprises an activated ester.

9. The method of any one of the preceding claims, wherein the controlled radical polymerization initiator comprises an alkyl halide or a chain transfer agent.

10. The method of any one of the preceding claims, wherein the controlled radical polymerization initiator is a compound of Formula (I): 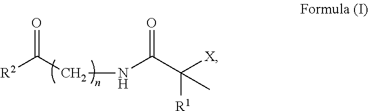
wherein:
X is a halogen or a chain transfer agent;
R¹ is hydrogen or alkyl;
R² is an active ester moiety; and
n is an integer from 1 to 6.

11. The method of claim 10, wherein X is Cl, Br or F.

12. The method of any one of the preceding claims, wherein the controlled radical polymerization initiator is a compound of Formula (II): 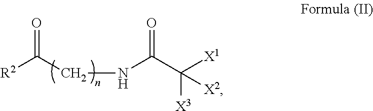
wherein:
X¹ is halogen or a chain transfer agent;
X² is methyl, phenyl, halogen or a chain transfer agent;
X³ is hydrogen, halogen or alkyl;
R² is an active ester moiety; and
n is an integer from 1 to 6.

13. The method of claim 12, wherein X¹ is Cl, Br or F.

14. The method of claim 12 or 13, wherein X² is Cl, Br or F.

15. The method of any one of the preceding claims, wherein each reaction chamber contains the same biomolecule.

16. The method of any one of the preceding claims, wherein the concentration of the controlled radical polymerization initiator is variable across the plurality of reaction chambers.

17. The method of any one of the preceding claims, wherein the plurality of reaction chambers comprises at least two different radical polymerization initiators.

18. The method of any one of claims 1 to 16, wherein each reaction chamber contains the same controlled radical polymerization initiator.

19. The method of any one of the preceding claims, wherein the biomolecule is immobilized in the reaction chamber.

20. A method of screening a plurality of biomolecule-polymer conjugates, the method comprising:
(a) providing a biomolecule-initiator conjugate, a monomer, and a catalyst to each reaction chamber in a plurality of reaction chambers;
(b) maintaining the plurality of reaction chambers under controlled radical polymerization conditions suitable for forming a plurality of biomolecule-polymer conjugates;
(c) simultaneously purifying each biomolecule-polymer conjugate in the plurality of biomolecule-polymer conjugates; and
(d) evaluating one or more properties of the purified biomolecule-polymer conjugates.

21. The method of claim 20, wherein the controlled radical polymerization conditions comprise conditions for an atom transfer radical polymerization (ATRP) procedure or a reversible-addition fragmentation chain transfer (RAFT) procedure.

22. The method of claim 20 or 21, wherein polymerization is induced by photoirradiation.

23. The method of claim 22, wherein each chamber is irradiated separately, and wherein duration and intensity of the photoirradiation is variable across the plurality of reaction chambers.

24. The method of any one of claims 20 to 23, wherein the providing of (a) further comprises removing oxygen from the plurality of reaction chambers.

25. The method of any one of claims 20 to 23, wherein the providing of (a) comprises:
(i) combining the biomolecule-initiator conjugate and the monomer in a buffer, thereby forming a mixture;
(ii) removing oxygen from the mixture; and
(iii) generating an active catalyst species in the deoxygenated mixture.

26. The method of any one of claims 20 to 25, wherein the catalyst maintains catalytic activity in the presence of oxygen.

27. The method of claim 24 or 25, wherein oxygen is removed by an enzyme-catalyzed reaction.

28. The method of claim 27, wherein the enzyme-catalyzed reaction comprises one or more enzymes selected from glucose oxidase, bilirubin oxidase, catechol dioxygenase, and luciferase.

29. The method of any one of claims 20 to 28, further comprising mixing the biomolecule-initiator conjugate, the monomer, and the catalyst in at least one of the reaction chambers in the plurality, thereby forming a homogenous mixture.

30. The method of any one of claims 20 to 29, wherein a polymer of the biomolecule-polymer conjugate formed by polymerization of the monomer is stimuli responsive.

31. The method of claim 30, wherein the stimuli is at least one of pH, temperature, or light.

32. The method of any one of claims 20 to 31, wherein the concentration of the monomer is variable across the plurality of reaction chambers.

33. The method of any one of claims 20 to 32, wherein the concentration of the catalyst is variable across the plurality of reaction chambers.

34. The method of any one of claims 20 to 33, wherein the identity of the catalyst is variable across the plurality of reaction chambers.

35. The method of any one of claims 20 to 34, wherein the biomolecule-initiator conjugate is immobilized in the reaction chamber.

36. The method of any one of claims 20 to 35, wherein the monomer is selected from a (meth)acrylate and a (meth)acrylamide.

37. The method of any one of claims 20 to 35, wherein the monomer comprises a mixture of at least two monomers selected from a (meth)acrylate and a (meth)acrylamide.

38. The method of any one of claims 20 to 37, further comprising, prior to the purifying of (c), providing a second monomer to the plurality of biomolecule-polymer conjugates.

39. The method of claim 38, wherein the monomer is selected from a (meth)acrylate and a (meth)acrylamide.

40. The method of any one of claims 36 to 39, wherein at least one of the (meth)acrylate and the (meth)acrylamide comprises one or more of a carboxybetaine, a sulfonate, a quaternary ammonium, a dialkylamino, an amino, a carboxylate, a hydroxyl, a sulfoxy or an oligo(ethylene glycol) moiety.

41. The method of any one of claims 20 to 40, wherein the monomer comprises a (meth)acrylate or a (meth)acrylamide, wherein the (meth)acrylate or the (meth)acrylamide comprises at least one of a sulfonate anion and an ammonium cation.

42. The method of any one of the preceding claims, wherein the biomolecule-initiator conjugate comprises a peptide or a protein.

43. The method of any one of the preceding claims, wherein the biomolecule-initiator conjugate is a compound of Formula (III): 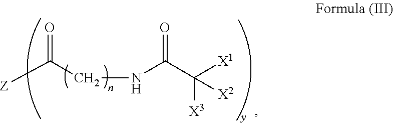
wherein:
Z is the biomolecule;
y is an integer from 1 to 100;
X¹ is halogen or a chain transfer agent;
X² is methyl, phenyl, halogen or a chain transfer agent;
X³ is hydrogen, halogen or alkyl;
R² is an active ester moiety; and
n is an integer from 1 to 6.

44. The method of any one of the preceding claims, wherein each reaction chamber in the plurality of reaction chambers is independently addressable by an automated liquid handling device.

45. The method of any one of the preceding claims, wherein the plurality of reaction chambers is on a single plate.

46. The method of any one of claims 1 to 44, wherein the plurality of reaction chambers is located on one or more plates.

47. The method of claim 45 or 46, wherein each reaction chamber on the plate comprises a membrane at the bottom of the reaction chamber.

48. The method of claim 47, wherein the membrane is an ultrafiltration membrane.

49. The method of claim 47 or 48, wherein the membrane is configured to allow continuous fluid delivery through the membrane.

50. The method of any one of claims 45 to 49, wherein the plate comprises at least 24 reaction chambers.

51. The method of any one of the preceding claims, wherein the plurality of reaction chambers comprises at least 24 reaction chambers.

52. The method of claim 50, wherein the plate comprises at least 96 reaction chambers.

53. The method of any one of the preceding claims, wherein the plurality of reaction chambers comprises at least 96 reaction chambers.

54. The method of any one of the preceding claims, wherein the purifying comprises ultrafiltration.

55. The method of claim 54, wherein the ultrafiltration is vacuum-assisted.

56. The method of any one of the preceding claims, wherein the purifying is accomplished with less than 1 mL of liquid per reaction chamber.

57. The method of any one of the preceding claims, wherein each reaction chamber in the plurality of reaction chambers is configured such that absorbance or fluorescence of the purified conjugates can be accurately measured by a spectrophotometer.

58. The method of any one of the preceding claims, wherein the evaluating comprises ultraviolet-visible spectroscopy, fluorescence spectroscopy or near-infrared spectroscopy.

59. The method of any one of the preceding claims, wherein the evaluating comprises assessing size of the purified conjugates.

60. The method of claim 59, wherein the assessing comprises one or more of size exclusion chromatography, mass spectrometry and dynamic light scattering.

61. The method of any one of the preceding claims, wherein the evaluating comprises assessing enzymatic activity of the purified conjugates.

62. The method of claim 61, wherein the enzymatic activity is assessed under normal working conditions of the biomolecule or under stress conditions.

63. The method of claim 62, wherein the stress conditions comprise, relative to normal working conditions, elevated or reduced temperature, elevated or reduced pH, or an elevated or reduced concentration of water in a buffer solution.

64. A library of biomolecule-initiator conjugates prepared according to the method of claim 1.

65. A library of biomolecule-polymer conjugates prepared according to the method of claim 20.

66. A library of biomolecule-polymer conjugates prepared by photoinduced atom transfer radical polymerization.

67. A library of biomolecule-polymer conjugates prepared by oxygen-tolerant photoinduced atom transfer radical polymerization.

68. A method of simultaneously isolating a plurality of bioconjugates from a plurality of reaction mixtures, the method comprising simultaneously passing a plurality of reaction mixtures comprising a plurality of bioconjugates through a plurality of ultrafiltration membranes, wherein the bioconjugates are retained above the membranes, the bioconjugates comprise a biomolecule conjugated to a controlled radical polymerization initiator or a biomolecule conjugated to a synthetic polymer, and wherein each reaction mixture in the plurality is independently purified.

69. A system for concurrently synthesizing a plurality of biomolecule-polymer conjugates, the system comprising:
(a) a plurality of reaction chambers configured to hold 1 to 1000 μL of fluid and to allow measurement of absorbance or fluorescence, by a spectrophotometer, of a biomolecule-polymer conjugate contained in each reaction chamber in the plurality;
(b) an automated device configured to deliver one or more of a reactant, solvent or catalyst to each reaction chamber in the plurality;
(c) optionally, an agitation module configured to mix contents of each reaction chamber in the plurality;
(d) a monitoring module configured to monitor progress of a reaction occurring in a reaction chamber in the plurality, wherein the monitoring module is in communication with a spectrophotometer configured to measure at least one of absorbance and fluorescence of the contents of at least one reaction chamber in the plurality;
(e) a purification module in fluid communication with the plurality of reaction chambers, wherein the purification module is configured to separate a biomolecule-polymer conjugate from other reaction mixture components, and wherein the other reaction mixture components comprise buffer, monomers and a catalyst; and
(f) an evaluation module in visual communication with the plurality of reaction chambers, wherein the evaluation module is configured to assess one or more physical properties of a biomolecule-polymer conjugate contained in each reaction chamber in the plurality.

70. The system of claim 69, further comprising a photoirradiation module in visual communication with the plurality of reaction chambers, wherein the photoirradiation module is configured to initiate, by photoirradiation, a polymerization reaction in a reaction chamber in the plurality.

71. The system of claim 70, wherein the photoirradiation module is configured to separately control the duration of photoirradiation for each of the plurality of reaction chambers.

72. The system of claim 70 or 71, wherein the photoirradiation module is configured to separately control the intensity of photoirradiation for each of the plurality of reaction chambers.

73. The system of any one of claims 69 to 73, further comprising a temperature control module configured to maintain the plurality of reaction chambers within a specific temperature range.

74. The system of claim 73, wherein the temperature control module comprises a coolant.
